(19) United States
(12) Patent Application Publication
(10) Pub. No.: US 2003/0049701 A1
(43) Pub. Date: Mar. 13, 2003

(54) Oncology tissue microarrays

(76) Inventor: Patrick J. Muraca, Pittsfield, MA (US)

Correspondence Address: PALMER & DODGE, LLP, PAULA CAMPBELL EVANS, 111 HUNTINGTON AVENUE, BOSTON, MA, 02199, US

(21) Appl. No.: 09/966,373

(22) Filed: Sep. 28, 2001

Related U.S. Application Data
(60) Provisional application No. 60/236,649, filed on Sep. 29, 2000.

(51) Int. Cl. G01N 33/574; G01N 1/30; G01N 33/48

(52) U.S. Cl. 435/7.23; 435/287.2; 435/40.5

(57) ABSTRACT

The invention provides oncology tissue microarrays. In one aspect, the microarrays comprise a plurality of cell and/or tissue samples, each sample representing a different type of cancer. In another aspect of the invention, each sample represents a different stage of cancer. In still a further aspect of the invention, samples are ordered on the substrate of the microarray into groups according to common characteristics of the patients from whom the samples are obtained. By dividing tissue samples on the substrate into different groupings representing different tissue types, subtypes, histological lesions, and clinical subgroups, the microarrays according to the invention enable ultra-high-throughput molecular profiling

### FIELD OF THE INVENTION

The invention relates to microarrays comprising a plurality of tissue samples for comparison to test tissue samples. The microarrays enable a user to evaluate disease progression and the likelihood of disease reoccurrence in a patient. In particular, the invention relates to microarrays comprising tissues representing a plurality of different stages of cancer.

### BACKGROUND OF THE INVENTION

The ability to monitor disease progression is an important tool in cancer therapy because it allows an attending physician to select the most appropriate course of treatment. For example, patients who are likely to relapse should be treated aggressively with powerful systemic chemotherapy and/or radiation therapy, while patients who are less likely to relapse can be treated less aggressively. Because using more aggressive therapeutic regimens can cause severe patient distress, it is desirable to determine whether a patient actually requires such aggressive treatment.

It The characteristic morphology of normal cells and tissues is ordinarily not preserved when they are transformed. Therefore, most, if not all, tumors can be identified and distinguished from normal tissues on the basis of histology. A cancerous tissue cell will typically lose morphological features in a process of dedifferentiation and will not maintain appropriate tissue boundaries (e.g., proliferating and invading regions where it would not normally be found). However, it is generally not possible on the basis of morphology alone to predict the likelihood that a given tumor cell will respond to a given therapeutic regimen or to determine whether the disease is likely to recur after treatment.

Molecular medicine aims to address the shortcomings of basic histology by providing information on the expression of specific gene products or variant gene products within a tissue sample. The expression or form of a gene product can be used as a marker if it appears characteristically when a phenotype such as disease (e.g., cancer) is observed. Numerous gene products have been shown to participate in or to be associated with human disease, and their measurement can provide diagnostic and prognostic tools to the clinician. However, the knowledge that a particular gene product is overexpressed in a tumor relative to a normal tissue still does not necessarily provide a predictive advantage if there is a continuum of harmful effects relating to varying levels of the gene product. For example, a range of levels of expression 1-5 of a gene product might be associated with phenotype A in which a cancer cell is relatively differentiated and respects its normal tissue boundaries, while an overlapping range of levels of expression, 4-8 would be associated with a phenotype B, in which a cancer cell is relatively undifferentiated but has not yet metastasized. At the boundaries between phenotypes (e.g., where the gene is expressed at levels 5-6), it is particularly difficult to make an accurate prognosis. The situation is complicated by the fact that a disease, such as cancer, represents the interactions of multiple genes, each of which may be expressed at varying levels.

By increasing the numbers of markers measured simultaneously, the accuracy of a prognosis can be improved. For example, Levine et al. (U.S. Pat. No. 5,843,684) describes a method of diagnosing or predicting the prognosis of cancer based on the co-expression of elevated levels of p53 and dm2. Patient samples are categorized into one of three groups depending upon whether either gene or both is expressed at an elevated level relative to normal tissue. Kamb et al. (U.S. Pat. No. 5,998,136) describes methods of identifying cell proliferation genes and methods of diagnosing or prognosing of diseases affecting cell proliferation based on altered expression of cell proliferation genes relative to normal tissues. Kallioniemi et al. (WO 00/24940) describes the use of tissue arrays to determine the correlation of genetic marker expression with various stages of disease.

In addition to the use of prognostic markers to inform the decision making of a physician, such markers serve as targets for drug development where a causal relationship exists between the marker and the disease. Due to the masses of genomic data being acquired in the human genome project, genomics-based drug development is now dependent upon the ability to rapidly prioritize and effectively exploit the new research leads identified. While hundreds of thousands of potential markers have been identified, information relating to the biological role of these markers is limited. The ability of pharmaceutical companies to develop new drugs and clinical products is becoming dependent upon their ability to choose the right targets out of these hundreds of thousands of targets and then to validate the chosen targets in vivo. This is where the current bottleneck lies. The drug developer must find a way to prioritize and select the most promising targets for further studies, be able to abandon the less promising targets as early as possible, and finally, to develop clinical and phenotypic information based on the characteristics of a target gene product at a population scale.

### SUMMARY OF THE INVENTION

There is a need in the art for new diagnostic and prognostic markers for disease. In addition, there is a need in the art for ways to correlate the expression characteristics of prognostic markers, new and old, with the severity of disease and the anticipated success of a range of treatment approaches. The microarrays and methods according to the present invention can be used to inform the decision making of clinicians with regard to the treatment of diseases, particularly cell proliferative diseases, such as cancer. The microarrays and methods according to the present invention further can be used to prioritize and validate drug targets identified in high throughput genomic screening techniques.

In one aspect of the invention, a microarray is provided comprising a plurality of tissue and/or cell samples, each sample stably associated with a different sublocation on the array, and comprising at least one known biological characteristic. The microarray can comprise from about 2 to greater than about 2000 sublocations. Preferably, at least 50% of the sublocations comprise different tissue types. In one aspect of the invention, at least one sublocation comprises human cells. In another aspect of the invention, at least one sublocation of the microarray comprises a cancer cell. Preferably, the microarray comprises a plurality of different types of cancer (e.g., from different tissues), while in another aspect of the invention, different grades of the same cancer are provided on the same microarray. More preferably, at least one sublocation comprises a healthy or normal tissue or cell sample.

The microarrays according to the present invention provide, on a single substrate, DNA, RNA, protein, and other biomolecule arrays as contained with cells and/or tissues. Detection of each of these different types of molecules may be performed optimally under different conditions. For example, while paraffin embedded sections may provide good morphology, such sections may not be optimal for detection of nucleic acids. Therefore, in one aspect of the invention, invention provides microarrays comprising heterogeneous sample types, such as paraffin-embedded or plastic-embedded tissue, frozen tissue, and a serum sample specimen, all on the same substrate and all from the same patient. In another aspect of the invention, sets of microarrays are provided comprising paraffin-embedded or plastic-embedded sections, frozen sections, and serum sample specimens all from the same patient. In a further aspect, a plurality of sets are provided representing a population of patients. Alternatively a single substrate can represent a “population microarray” comprising cells and/or tissues from a plurality of individuals.

The invention further provides a method for comparing the biological characteristics of a test tissue or cell(s) (“a test sample”), comprising providing a test sample, contacting the test sample with a molecular probe, and identifying the reactivity of the molecular probe with the test sample and cell and/or tissue samples within a microarray. In one aspect of the invention, reactivity is any of: specific labeling, binding, enzymatic catalysis, and/or hybridization. In another aspect of the invention, the reactivity of the test sample and cells and/or tissues on the microarray is correlated with information relating to the source of the cells and/or tissues on the microarray. Preferably, the information includes patient information. The reactivity of the sample also can be correlated with molecular profiling data which has been obtained for cells and/or tissues on the microarray.

In one aspect of the invention, a profile array substrate is provided comprising a first location for placement of a test tissue and a second location comprising a microarray. In this aspect, the biological characteristics of a test tissue can be evaluated at the same time and under the same conditions as the biological characteristics of the cells/tissues within the microarray.

In a preferred aspect of the invention, information relating to samples on the microarrays is stored in a specimen-linked database. Such information can include one or more of: gene expression (e.g., such as RNA expression, RNA processing; and protein expression, protein modifications, cleavage, and processing); expression of other cellular biomolecules, tissue type, disease status, and patient information (e.g., patient medical history, including drug exposure, age, sex, age and cause of death if appropriate, family medical history, and the like). In one aspect of the invention, the information is displayed on the display of a user computer or a wireless device connectable to a network. Preferably, as information relating to test samples is obtained, this information is also stored in the database. The specimen-linked database is also used to store information relating to correlations between biological characteristics of test samples and the biological characteristics of specimens for which data exists in the database (e.g., such as data obtained from one or more microarrays).

The microarrays according to the invention are used in methods of selecting promising gene targets, sorting/prioritizing cDNA array data, surveying entire populations, validating gene discoveries in 100's of human tissue specimens, investigating disease pathogenesis and progression, and searching for diagnostic, prognostic and clinical correlations, such as the likelihood of disease reoccurrence.

In one aspect of the invention, an oncology array is provided comprising different cell and/or tissue types from a patient with cancer, including cancerous cells (both primary and secondary sites of cancer), and normal cells. The patient may have additional characteristics affecting disease progression (e.g., age, exposure to an environmental condition, treatment with a drug, or radiation, one or more underlying or concurrent illnesses, and the like). The oncology microarray can be reacted with molecular probes to generate a diagnostic matrix which correlates information relating to the reactivity of the probes with other biological characteristics which identify that patient, such that reactivity of the molecular probes can then be used to predict the presence of these other biological characteristics in that patient.

In one aspect of the invention, the diagnostic matrix provides a correlation between the expression of cancer-specific gene and a particular stage of cancer, e.g., enabling a user to diagnose the progression of a cancer in a patient. Information relating to the expression of the cancer-specific gene in a test sample from a patient is then compared to information within the diagnostic matrix to identify the particular stage of cancer associated with that level of expression. This information can be used to provide a prognosis and to guide a physician as to what course of therapy to use in treating the patient.

In another aspect of the invention, a set of arrays is provided, each array representing a different patient. By increasing the number of biological characteristics and patients looked at, a highly informative database is generated which a user can access to evaluate disease progression, the efficacy of a particular treatment, and the effects of underlying conditions (e.g., such as age, other types of disease, etc). In one aspect, the database is used to prioritize drug targets. A plurality of disease microarrays, each representing different cells/tissues from different patients with diseases can be used to profile a known or unknown biological molecule, using the power of parallel analysis to determine the biological relevance of these molecules.

In a further aspect of the invention, samples within a microarray are ordered into groups which represent the patients from which these tissues are derived. In one aspect, the groupings are based on multiple patient parameters that can be reproducibly defined from the development of molecular disease profiles. A clinical diagnosis/prognosis can be made using any or all of the parameters identified.

### BRIEF DESCRIPTION OF THE DRAWINGS

The objects and features of the invention can be better understood with reference to the following detailed description and accompanying drawings.

FIG. 1A shows a flow chart according to one aspect of the invention in which microarrays according to the invention are used in conjunction with gene chips to identify, prioritize, and validate drug targets. FIG. 1B shows a schematic diagram of how data from a microarray is used in this process.

FIG. 2A is an illustration of a profile array substrate according to one aspect of the invention, comprising a first location for placing a test tissue sample and a second location comprising a microarray. The microarray comprises a plurality of sublocations, each sublocation comprising a sample stably associated therewith representing a different stage of breast cancer. FIG. 2B shows an array locator according to one aspect of the invention next to a profile array substrate for identifying samples which have reacted with molecular probes. FIG. 2C shows six different tissue samples representing different stages of breast cancer stained with a CK7 antibody. FIG. 2D shows information provided in a kit which comprises the profile array substrate shown in FIG. 2A and the array locator shown in FIG. 2B. FIG. 2E shows a profile array substrate comprising a test tissue at a first location and a microarray at a second location. The test tissue is stained with a breast cancer specific antibody.

FIG. 3 shows a tissue microarray according to the present invention comprising a plurality of sublocations, each sublocation comprising a tissue sample whose morphological features can be distinguished under a microscope.

FIGS. 4A-4C show an interface on a display of a user device connectable to a network which displays information relating to the biological characteristics of tissues at different sublocations on the microarray. FIG. 4A shows an interface for addressing a breast cancer microarray and for inputting information into a specimen-linked database comprising information relating to the biological characteristics of breast cancer tissues at different sublocations on the microarray. FIG. 4B shows a display of a portion of a specimen-linked database. FIG. 4C shows a display enabling a user to access a relational database for identifying relationships between biological characteristics of tissues at different sublocations on the array.

### DESCRIPTION

The invention provides oncology microarrays which comprise a substrate on which a plurality of tissue and/or cell samples are provided, each sample being stably associated with the substrate at a different, known, position on the substrate. Preferably, samples represent different types or stages of cancer. Samples can be ordered on the substrate of a microarray into groups according to common characteristics of the patients from whom the samples are obtained (e.g., a group of samples from patients treated with chemotherapy, a group of samples from patients not treated with chemotherapy, a group from patients treated with hormones, a group from patients not treated with hormones, etc.). By dividing samples on the substrate into different groupings representing different cell/tissue types, subtypes, histological lesions, and clinical subgroups, the microarrays according to the invention enable ultra-high-throughput molecular profiling

### DEFINITIONS

The following definitions are provided for specific terms which are used in the following written description.

As used herein, the term “biological characteristics” refers to the phenotype and/or genotype of one or more cells or tissues being arrayed and can include one or more of cell type tissue type, morphological features of the cells and/or tissues, and the expression of biological molecules with the cells/tissues. The “expression of biological molecules” can include the expression ands accumulation of RNA sequences, the expression and accumulation of proteins (including the expression of their modified, cleaved, and/or processed forms, and further including the expression and accumulation of enzymes, their substrates, products and intermediates) as well as the presence or absence or copy number of particular chromosomes or chromosome regions within the cell. “Biological characteristics of a cell source” or “biological characteristics of tissue source” refers to the characteristic of the patient who is the source of the cells (e.g., such as the age, sex and physiological state of the organism) and encompasses patient information.

As used herein, “a diagnostic trait” is an identifying characteristic which includes both biological characteristics and experiences (e.g., exposure to a drug). A trait can be a marker for a transformed, immortalized, pre-cancerous, or cancerous state, or a marker for a particular cell type, or the ability of a tissue to bind, incorporate, or respond to a drug or agent. A trait also refers to the response of a tissue or cell to a protein, drug, or other agent. In one aspect, a trait is a marker for a particular cell type such as a transformed, immortalized, pre-cancerous, or cancerous cell or a state, such as a disease, and detection of the train provides a reliable indicia that the sample comprises the cell(s). Screening or evaluating for a diagnostic trait thus refers identifying an agent which can cause a detectable change in a trait which is statistically significant when compared to cells not so treated using routing statistical tests. As used herein, a “trait” can be the expression of one or more biological characteristics.

As defined herein, a “molecular probe” is any detectable molecule or molecule which produces a detectable molecule upon reacting with a biological molecule. “Reacting” encompasses binding, labeling, or catalyzing an enzymatic reaction. A “biological molecule” is any molecule which is found in a cell or within the body of an organism.

As used herein, the term “substantially matches”, when referring to an expression characteristic, means that the score assigned to a patient's tissue sample for a given polypeptide using a scoring method as described herein is the same (which is defined as not being significantly different using routine statistical tests to within 95% confidence levels) as the score for a tissue sample to which it is being compared for at least that polypeptide. The scoring methods useful in the invention assign a value to every expression characteristic, with each such value actually representing a range of values. Since both the patient sample and the standard samples are scored using the same method and the same ranges of values for each class, there will always be a substantial match between a patient sample and one or more tumor or normal samples on the panel, even though the level of expression does not exactly match between the respective samples.

As used herein, the term “expression” refers to the level, form or localization of a product. For example, the “expression of a protein” refers to one or more of the level, form (e.g., presence, absence, or amount of modifications, and/or presence, absence or amount of cleaved or otherwise processed products of a protein), or localization of the protein.

As used herein, the term “difference in biological characteristics” refers to an increase or decrease in a measurable expression of given biological characteristic. A difference may be an increase or decrease in a quantitative measure (e.g., an amount of protein or amount of RNA encoding the protein) or a change in a qualitative measure (e.g., the localization of an RNA or protein). Where a difference is observed in a quantitative measure, the difference according to the invention will be at least about 10% greater or less than the level in a normal standard sample (e.g., a control). Where a difference is an increase, the increase may be as much as about 20%, about 30%, about 50%, about 70%, about 90% to about 100% (about two-fold) or more, up to and including about 5-fold, 10-fold, 20-fold, 50-fold or more. Where a difference is a decrease, the decrease may be as much as about 20%, 30%, 50%, 70%, 90%, 95%, 100% (e.g., where there is no specific protein or RNA present). It should be noted that even a qualitative difference could be represented in quantitative terms if desired. For example, a change in the intracellular localization of a polypeptide may be represented as a change in the percentage of cells showing the original localization.

A “disease or pathology” is a change in one or more biological characteristics that impairs normal functioning of a cell, tissue, and/or organism. A “pathological condition” encompasses disease but also encompasses abnormal responses which are not associated with any particular infectious organism or single genetic alteration in an individual. For example, as defined herein, a stroke or immune response occurring after the transplantation of an organism would be encompassed by the term “pathological condition”.

As defined herein, “a cell proliferative disorder” is a condition marked by any abnormal or aberrant increase in the number of cells of a given type or in a given tissue. Cancer is often thought of as the prototypical cell proliferative disorder, yet disorders such as atherosclerosis, restenosis, psoriasis, inflammatory disorders, some autoimmune disorders (e.g., rheumatoid arthritis) are also caused by abnormal proliferation of cells, and are thus examples of cell proliferative disorders.

As used herein, “a tumor” is a neoplasm that may either be malignant or non-malignant. Tumors of the same tissue type originate in the same tissue, and may be divided into different subtypes based on their biological characteristics.

As used herein, the term “disease recurrence” refers to the development or emergence of cells of a proliferative disease, such as a tumor, after a treatment that has substantially removed such cells. A disease recurrence may be at the same site as the original disease or elsewhere, but will involve accumulation of cells of the same tissue of origin as in the original disease.

As used herein, the term “guiding treatment” refers to the process of informing the decision making for the treatment of a disease. As used herein, treatment guidance is based on the comparative levels of one or more cell growth-related polypeptides in a patient's tissue sample relative to the levels of the same polypeptide(s) in a plurality of normal and diseased tissue samples from individuals for whom patient information, including treatment approaches and outcomes is available.

The term “donor block” as used herein, refers to tissue that may be embedded in an embedding matrix, from which a tissue sample is obtained and placed directly onto a slide or placed into a receptacle of a recipient block.

The term “donor sample” as used herein, refers to a tissue sample obtained from the donor block.

The term “recipient block” as used herein, refers to a block formed from an embedding matrix, having receptacles to hold donor samples in a regular pattern so that the location of the donor samples will be maintained when the recipient block is sectioned to produce an array of tissue samples.

As used herein, the term “course of disease” refers to the sequence of events in which a disease develops, causes symptoms and is either recovered from or continues and/or increases in severity.

As used herein, the term “cancer” refers to a malignant disease caused or characterized by the proliferation of cells which have lost susceptibility to normal growth control. “Malignant disease” refers to a disease caused by cells that have gained the ability to invade either the tissue of origin or to travel to sites removed from the tissue of origin.

As used herein, the term “tumor suppressor gene” refers to a gene, the normal expression of which tends to prevent the establishment or growth of oncogenically transformed cells. A tumor suppressor gene may act, for example, to halt or slow the proliferation of cells or, for example, to cause the cells to undergo apoptosis in response to a particular stimulus or condition.

As used herein, the term “tumor stage” refers to a measure of the degree of advancement or progression of a tumor. A tumor's stage is determined according to criteria including, for example, the morphology of the cells, morphology of the tissue, whether tumor cells have infiltrated the tissue of origin, whether tumor cells have invaded lymph nodes, and whether distant metastasis has occurred. Clinical staging for many tumors follows the TNM system, described herein below, but other clinical staging scales adapted to specific diseases are known in the art.

As used herein, the term “non-tumor samples” refers to tissue samples obtained from normal tissue. A sample may be judged a non-tumor sample by one of skill in the art on the basis of morphology.

As used herein, the term “computer-accessible file” refers to a collection of information regarding a tissue sample, which collection is stored in a computer's memory and is retrievable or can be accessed through that computer or one linked to it.

As used herein, the term “information about the patient” refers to any information known about the individual from whom a tissue sample was obtained. Examples of information that would be useful in a given file of information about the patient include age, sex, weight, height, ethnic background, family medical background, the patient's medical history (e.g., information pertaining to prior cell proliferative disorders, infectious diseases and metabolic disorders, diagnostic and prognostic test results, drug exposure, responses to drug exposure, other treatment approaches and their success or failure, cause of death, etc.).

As used herein, the term “degree of disease severity” refers to measure of how advanced a disease is, on a scale from no disease to the worst possible disease. One of skill in the art can place a set of tissue samples representing a disease in order of ascending or descending severity of disease. In order to do so, samples may be compared not only to known standards, but also to each other.

As used herein, the term “detectable binding reagent” refers to an agent that specifically recognizes and interacts or binds with an entity one wishes to measure, wherein the agent has a property permitting detection when bound. “Specifically interact” means that a binding agent physically interacts with the entity one wishes to measure, to the exclusion of other entities also present in the sample. The binding of a detectable binding reagent useful according to the invention has stability permitting the measurement of the binding. A detectable binding reagent may possess an intrinsic property that permits direct detection, or it may be labeled with a detectable moiety.

As used herein, the term “detectable moiety” refers to a moiety that can be attached to a binding reagent that confers detection of the binding reagent by a particular method or methods. Detectable moieties include, but are not limited to radiolabels (e.g., ³²p, ³⁵S, ¹²⁵I, etc.), enzymes (e.g., alkaline phosphatase, peroxidase, etc.), fluorophores (e.g., fluorescein, amino coumarin acetic acid, tetramethylrhodamine isothiocyanate (TRITC), Texas Red, Cy3.0, Cy5.0, green fluorescent protein, etc.) and colloidal metal particles.

As used herein, the term “labeled” means that a detectable moiety has been physically attached to a binding reagent.

As used herein, the term “antibody or antigen binding fragment thereof” refers to an immunoglobulin having the capacity to specifically bind a given antigen. The term “antibody” as used herein is intended to include whole antibodies of any isotype (IgG, IgA, IgM, IgE, etc), and fragments thereof which are also specifically reactive with a vertebrate, e.g., mammalian, protein. Antibodies can be fragmented using conventional techniques and the fragments screened for utility in the same manner as whole antibodies. Thus, the term includes segments of proteolytically-cleaved or recombinantly-prepared portions of an antibody molecule that are capable of selectively reacting with a certain protein. Non-limiting examples of such proteolytic and/or recombinant fragments include Fab, F(ab′)2, Fab′, Fv, and single chain antibodies (scFv) containing a V[L] and/or V[H] domain joined by a peptide linker. The scFv's may be covalently or non-covalently linked to form antibodies having two or more binding sites. Antibodies may be labeled with detectable moieties by one of skill in the art. In some aspects, the antibody that binds to an entity one wishes to measure (the primary antibody) is not labeled, but is instead detected by binding of a labeled secondary antibody that specifically binds to the primary antibody.

As defined herein, a “nucleic acid array,” “peptide array”, “a polypeptide array”, “protein array” or “small molecule” array refers to a plurality of nucleic acids, peptides, polypeptides, proteins, or small molecules, respectively that are immobilized on a substrate in different, known locations on the substrate.

As defined herein, a “tissue” is an aggregate of cells that perform a particular function in an organism. The term “tissue” as used herein refers to cellular material from a particular physiological region. The cells in a particular tissue may comprise several different cell types. A non-limiting example of this would be brain tissue that further comprises neurons and glial cells, as well as capillary endothelial cells and blood cells, all contained in the tissue section.

As defined herein a “cell and/or tissue microarray” is a microarray which comprises a plurality of sublocations, each sublocation comprising one or more cell(s) or a portion of a tissue stably associated with a substrate at that sublocation. The term “microarray” implies no upper limit on size on samples in the array but merely encompasses a plurality of samples stably associated with distinct, known sublocations on a substrate, which, in one aspect, can be viewed using a microscope.

As used herein, a sample or portion of a sample which is “stably associated with a substrate” refers to a sample or portion thereof which does not substantially move from its position on the substrate during one or more molecular procedures.

As used herein “molecular procedure” refers to contact with a test reagent or molecular probe such as an antibody, nucleic acid probe, enzyme, chromagen, label, and the like. In one aspect, a molecular procedure comprises a plurality of hybridizations, incubations, fixation steps, changes of temperature (from −4° C. to 100° C.), exposures to solvents, and/or wash steps

As used herein, “a whole body microarray” is a microarray comprising cell and/or tissue samples representing the whole body of an organism. In one aspect, the microarray comprises at least about five different types of cells/tissues from a single organism, at least about ten different types of cells/tissues or at least about 20 different types of cells/tissues from the organism, each different type of cell/tissue stably associated with a different, distinct, known sublocation of the microarray. As used herein, “different types of cells” refer to cells which differ in the expression of a least one peptide, polypeptide, or protein. Preferably, different types of cells and/or tissues are from different organs or are from anatomically and/or histologically distinct sites in the same organ. For example, in one aspect, a whole body microarray comprises at least five different types of tissues selected from the group consisting of brain, cardiac tissue, liver, pancreas, spleen, stomach, lung, skin, eye, colon, reproductive organ (male or female) and kidney cells. In preferred aspects, a sample of cells from a bodily fluid is also included such as a blood sample, lymph sample, CSF sample, urine sample, amniotic fluid sample, leukapheris sample, and the like. Cells can also be selected from the group consisting of hematopoietic cells, stem cells and progenitor cells, T cells, B cells, monocytes, granulocytes, dendritic cells, macrophages, erythroid cells, mekaryocytes, platelets, endothelial cells, epithelial cells, tumor cells, fibroblasts and the like.

As used herein “substantially identical microarrays” refer to microarrays obtained by sectioning a single microarray block. Preferably, substantially identical microarrays comprise sections which are within about 0-500 μm of each other in a microarray block. Substantially identical microarrays comprise a one-to-one correspondence of samples, such that samples at identical coordinates in a plurality of substantially identical microarrays will be substantially identical (e.g., express substantially the same biomolecules and have substantially the same morphological features).

As defined herein, a “database” or a “specimen-linked database” refers to a collection of information or facts organized according to a data model which determines whether the data is ordered using linked files, hierarchically, according to relational tables, or according to some other model determined by the system operator. The organization scheme the database uses is not critical to performing the invention, so long as information within the database is accessible to the user through an information management system. Data in the database are stored in a format consistent with an interpretation based on definitions established by the system operator (i.e., the system operator determines the fields which are used the define patient information, molecular profiling information, or another type of information category. Preferably, a “specimen-linked database’ is one which cross-references information in the database to specimens provided on one or more microarrays, and preferably using codes such as SNOMED codes, ICD-9 codes, and/or DSM-IV TR codes.

As used herein, “a system operator” is one who controls access to the database.

As used herein, the term “information management system” refers to a system which comprises a plurality of functions for accessing and managing information within the database. Minimally, an information management system according to the invention comprises a search function for locating information within the database and for displaying at least a portion of the information to a user, and a relationship-determining function, for identifying relationships between information or facts stored in the database.

As used herein, “an interface” or “user interface” or “graphical user interface” is a display comprising text and/or graphical information displayed by the screen or monitor of a user device connectable to the network (e.g., such as the world wide web) which enables a user to interact with the specimen-linked database and information management system.

As used herein, the term “link” refers to a point-and-click mechanism implemented on a user device connectable to the network which enables a user/viewer to link (or jump) from one display or interface where information is referred to (“a link source”) to other screen displays where information exists (a “link destination”). The term “link” encompasses both the display element that indicates the information is available and the program which finds the information (e.g., within the database) and displays it on the destination screen). In one aspect, a link is associated with text; however, in other aspects, links are associated with images or icons. In some aspects, selecting a link (e.g., by right clicking on a mouse) will cause a drop-down menu to be displayed which provides the user with the option of viewing one of several interfaces. Links also can be provided in the form of action buttons, radio buttons, check buttons, and the like.

As defined herein, a “browser” is a program which supports the displaying of documents across a network. Browsers enable accessing linked information over the Internet and other networks as well as from magnetic disks, CD-ROMs, or other memory sources”

The term “providing access to a database” or “providing access to a portion of a database” as defined herein refers to making information in the database available to end users in a usable format. A usable format depends on the capabilities and needs of the user, and the complexity and volume of the information, and may be for example, written text, an image, a combination of written and visual information, verbal, by electronic means, including a computer readable format (e.g., software, optical discs, and the light), or any other format for conveying information such as text or visual data. The term “providing access” should not be necessarily construed as providing full access to all records in a database, or that all tissues/cells on a microarray have records in the database.

The term “research report” as used herein refers to report or analytical summary of the information obtained during the process of providing a microarray according to the invention, and providing access to a specimen-linked database. The report or analysis is intended to reflect the needs of the clients. The report may be provided in written format, electronic format such as, for example, electronic mail, or contained on a magnetic storage device such as a computer disk or tape, by facsimile, verbally, or by telephone, or by written or visual or any other means.

The term “analysis” as used herein refers to any scientific, medical, or general use of the tissue microarray and/or the database for the purpose of obtaining information or data, including tissue identification data. This term is intended to cover any of the techniques disclosed in this application. Likewise, as scientific techniques are under constant refinement, it also comprehends the use of other manipulations or experimentation that involve the investigation of nucleic acids or proteins of the tissues on the tissue microarray.

As defined herein, “an individual” is a single organism and includes humans, animals, plants, multicellular and unicellular organisms.

“High throughput techniques” are techniques that evaluate large numbers (at least 10) of samples at a single time.

As used herein, a “correlation” refers to a statistically significant relationship using routine statistical methods known in the art. For example, in one aspect, statistical significance of a correlation is determined using a Student's unpaired t-test, considering differences as statistically significant at p<0.05.

As used herein, a “diagnostic probe” is probe whose reactivity with a sample provides an indication of the presence or absence of a diagnostic trait. In one aspect, a probe is considered to be diagnostic if it binds to a diseased cell and/or cells in at least about 80% of a plurality of samples comprising diseased cells (“disease samples”) and binds to less than 10% of non-disease cell(s). Preferably, the probe binds to at least about 90% or at least about 80% of disease samples and binds to less than about 5% or less than about 1% of non-disease samples.

Microarrays

The microarrays according to the invention comprise a plurality of sublocations, each sublocation comprising a cell or tissue sample having at least one known biological characteristic (e.g., such as cell or tissue type) which is stably associated with a substrate at the sublocation. In a preferred aspect of the invention, the plurality of sublocations comprise cancerous tissue at different neoplastic stages. The sublocations are distinct from each other in that they are separated by regions of substrate with no sample stably associated therewith.

Substrates

The substrate facilitates handling of the microarray through a variety of molecular procedures. In one aspect of the invention, the microarray substrate is solvent resistant. In another aspect of the invention, the substrate is transparent. In still another aspect of the invention, the microarray substrate comprises any of: glass; quartz; fused silica; or other nonporous substrate; plastic, such as polyolefin, polyamide, polyacrylamide, polyester, polyacrylic ester, polycarbonate, polytetrafluoroethylene, polyvinyl acetate, and/or can comprise a plastic composition containing fillers (such as glass fillers), extenders, stabilizers, and/or antioxidants; celluloid, cellophane or urea formaldehyde resins ,or other synthetic resins such as cellulose acetate ethylcellulose, or other transparent polymer.

In one aspect, the microarray substrate is rigid; however, in another aspect, the substrate is semi-rigid or flexible (e.g., a flexible plastic comprising polycarbonate, cellular acetate, polyvinyl chloride, and the like). In a further aspect, the substrate is optically opaque and substantially non-fluorescent. Nylon or nitrocellulose membranes also can be used as substrates and can include materials such as polycarbonate, polyvinylidene fluoride (PVDF), polysulfone, mixed esters of cellulose and nitrocellulose, and the like.

The size and shape of the substrate may generally be varied. However, preferably, the substrate fits entirely on the stage of a microscope. In one aspect, the substrate is planar. In another aspect, the substrate is about 1 inch by 3 inches, 77×50 mm, or 22×50 mm. In a further aspect of the invention, the microarray substrate is at least about 10-200 mm×10-200 mm.

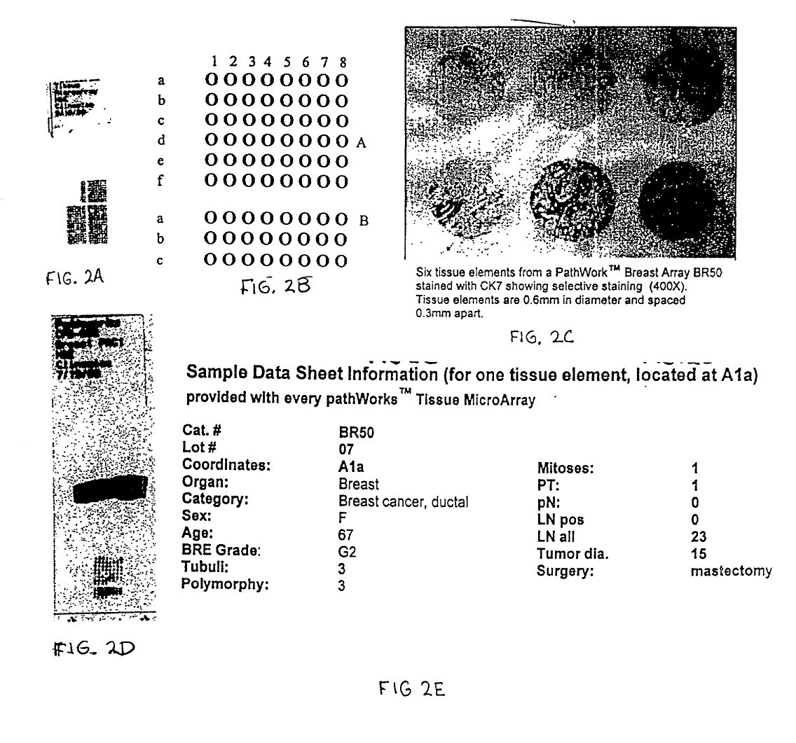

As shown in FIG. 2B, the substrate also can be configured as a profile array substrate designed to accommodate a control microarray (a microarray comprising cell and/or tissue samples for which at least one biological characteristic being assayed for is known) and a test sample for comparison with the control microarray. Profile array substrates generally comprise a first location for placing a test sample and a second location comprising the microarray. In this aspect, the first location is for placing a test tissue sample while the second sublocation comprises the microarray. The profile array substrate allows testing of a test tissue sample to be done simultaneously with the testing of samples on the control microarray allowing for a side-by-side comparison of biological characteristics of the test sample with the characteristics of the cells/tissues in the microarray.

Additional Features of the Substrate

In one aspect of the invention, the substrate comprises a location for placing an identifier (e.g., a wax pencil or crayon mark, an etched mark, a label, a bar code, a microchip for transmitting radio or electronic signals, and the like) which provides a user of the microarray with access to a specimen-linked database comprising information relating to one or more samples (and preferably, all) of the microarray. Where the identifier is microchip, the microchip communicates with a processor which comprises or can access the specimen-linked database.

Sublocations

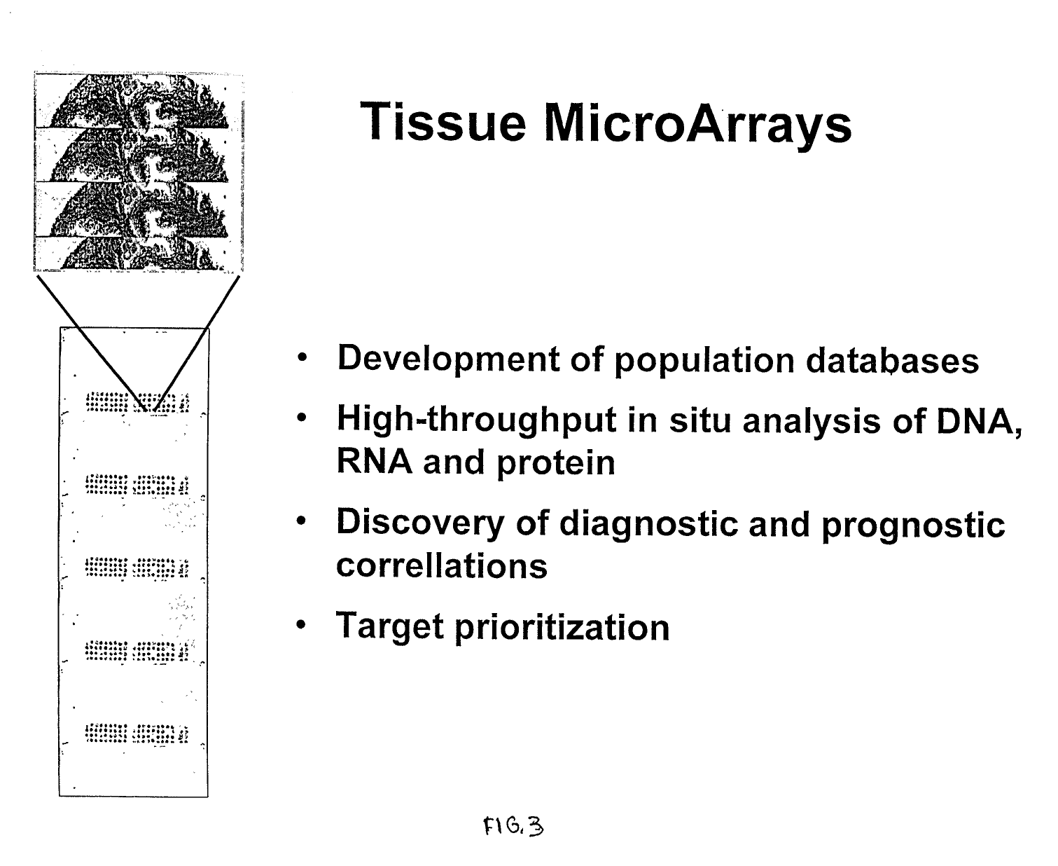

Each sublocation of the microarray comprises cell(s) or a tissue sample stably associated therewith. In one aspect, the cells/tissue have morphological features substantially intact which can be at least viewed under a microscope to distinguish subcellular features (e.g., such as a nucleus, an intact cell membrane, organelles, and/or other cytological features), i.e., the sample is not lysed (see, as shown in FIG. 3).

In one aspect of the invention, the microarray comprises from about 2-1000 sublocations. In another aspect, the microarray comprises about 2 sublocations, about 5 sublocations, about 10 sublocations, about 20 sublocations, about 25 sublocations, about 30 sublocations, about 45 sublocations, about 50 sublocations, about 55 sublocations, about 60 sublocations, about 65 sublocations, about 75 sublocations, about 100 sublocations, about 150 sublocations, about 200 sublocations, about 250 sublocations, or about 500 sublocations, about 550 sublocations, about 600 sublocations, about 650 sublocations, about 700 sublocations, about 750 sublocations, about 800 sublocations, about 850 sublocations, about 900 sublocations, about 950 sublocations, or about 1000 sublocations. In one aspect of the invention, each sublocation is from about 2-10 mm apart. In another aspect of the invention, each sublocation comprises at least one dimension which is about 0.3 μm-20 mm. The sublocations can be organized in any pattern, and each sublocation can be generally any shape (square, circular, oval, elliptical, disc-shaped, rectangular, triangular, and the like).

In a preferred aspect, the sublocations are positioned in a regular repeating pattern (e.g., rows and columns) such that the identification of each sublocation as to cell/tissue type can be ascertained by the use of an array locator (as shown in FIG. 2D). In one aspect, the array locator is a template having a plurality of shapes, each shape corresponding to the shape of each sublocation in the array and maintaining the same relationships as each sublocation on the array. The array locator is marked by coordinate, allowing the user to readily identify a sublocation on the array by virtue of unique coordinates. In one aspect of the invention, the array locator is a transparent sheet (e.g., plastic, acetate, and the like). In another aspect of the invention, the array locator is a sheet comprising a plurality of holes, each hole corresponding in shape and location to each sublocation on the array.

Oncology Microarrays

In a preferred aspect of the invention, a plurality of sublocations on the microarray comprise abnormally proliferating cells. In one aspect, the sublocations comprise one or more of: cells from a brain tumor, pituitary tumor, cancerous eye tissue (e.g., a retinoblastoma), cancerous tongue tissue, cancerous tracheal tissue, cancerous esophageal tissue, liver tumor, spleen tumor, a lymphoma, cancerous testicular or prostate tissue, cancerous cervical tissue, cancerous uterine tissue, cancerous bladder tissue, cancerous kidney tissue, cancerous thyroid, cancerous colon tissue, cancerous pancreatic tissue, cancerous skin tissue, cancerous breast tissue, cancerous stomach tissue. The microarray also can include cells from adenomas and sarcomas from various tissues.

In one aspect of the invention, each sublocation comprises the same cell or tissue type, to form a brain tumor array, a pituitary tumor array, a retinoblastoma array, cancerous tongue array, cancerous tracheal tissue array, cancerous esophageal tissue array, liver tumor array, cancerous spleen tissue array, lymphoma array, cancerous testicular tissue array, cancerous cervical tissue array, cancerous uterine tissue array, cancerous kidney tissue array, cancerous bladder tissue array, cancerous thyroid tissue array, cancerous prostate tissue array, cancerous colon tissue array, cancerous pancreas tissue array, cancerous breast tissue array, and cancerous stomach tissue array. Preferably, for each type of cancerous tissue in the microarray there is at least one noncancerous cell or tissue of the same type.

In one aspect of the invention, the microarray comprises at least one sublocation comprising cancerous cells including, but not limited to, breast ductal carcinoma, bladder carcinoma, leiomyoma, meningioma, melanoma, melanoma with a Clark score of 1-5 with nevus, seminoma, lymphoma, and colon adenocarcinoma, and any of the cancer types listed above.

In another aspect of the invention, the microarray comprises at least two sublocations comprising cells from different tissues. In one aspect of the invention, at least 50% of the sublocations in the microarray comprise cells from different tissues. In still a further aspect of the invention, at least 60%, 70%, 80%, 90%, or 100% of the array comprises cells from different tissues. Preferably, these different tissues are from the same patient.

In another aspect of the invention, the microarray comprises a plurality of sublocations, and at least one sublocation comprises at least about one, at least about five, at least about ten, substantially duplicate sublocations (e.g., comprising cells from the same tissue, and/or from the same representative area of a donor sample, as described further below).

In still another aspect, an oncology microarray is provided comprising at least about 10, at least 20, at least 30, at least 40, at least 50, at least 100, at least 150, or at least 200 different types.

In another aspect of the invention, at least one sublocation comprises cells from an individual with an enhanced cancer susceptibility (e.g., there is a family history of cancer or the patient has had cancer previously or has been or is being exposed to carcinogen(s)).

In a further aspect of the invention, the microarray comprises at least one sublocation comprising cells or tissue from an individual with a disease other than cancer or in addition to cancer (e.g., including, but not limited to: a blood disorder, blood lipid disease, autoimmune disease, bone or joint disorder, a cardiovascular disorder, respiratory disease, endocrine disorder, immune disorder, infectious disease, muscle wasting and whole body wasting disorder, neurological disorder, skin disorder, kidney disease caused by excessive fibrosis, scleroderma, stroke, hereditary hemorrhage telangiectasia, disorders associated with diabetes, hypertension, diabetes, manic depression, depression, borderline personality disorder, anxiety, schizophrenia, Gaucher disease, cystic fibrosis and sickle cell anemia, and the like). For further discussion of human genetic diseases, see Mendelian Inheritance in Man: A Catalog of Human Genes and Genetic Disorders by Victor A. McKusick (12th Edition (3 volume set) June 1998, Johns Hopkins University Press, ISBN: 0801857422), the entirety of which is incorporated herein.

Samples at individual sublocations of the microarrays also can be obtained from individuals exposed to the same environmental conditions (past or ongoing). For example, samples from patients exposed to carcinogens (known or suspected), pollutants, asbestos, and other agents also can be arrayed.

In a different aspect, a microarray is provided comprising cells from a plurality of individuals who have all died from the same pathology or from individuals being treated with the same drug (including those who recovered from the disease and/or those who did not).

In still a further aspect, samples can be obtained from patients comprising substantially the same molecular profile (e.g., expression of RNA and/or protein) with respect to one or more genes or sets of genes.

In a further aspect of the invention, each sublocation of the microarray comprises cells from different members of a pedigree sharing a family history of cancer (e.g., selected from the group consisting of sibs, twins, cousins, mothers, fathers, grandmothers, grandfathers, uncles, aunts, and the like). In another aspect of the invention, the “pedigree microarray “comprises environment-matched controls (e.g., husbands, wives, adopted children, stepparents, and the like). In still a further aspect of the invention, the microarray is a reflection of a plurality of traits representing a particular patient demographic group of interest, e.g., overweight smokers, diabetics with peripheral vascular disease, individuals having a particular predisposition to disease (e.g., sickle cell anemia, Tay Sachs, severe combined immunodeficiency), where individuals in each group have cancer.

In another aspect, an oncology microarray is provided comprising at least one sublocation comprising cells from a cell line of cancerous cells. In one aspect of the invention, the cell line is a continuous cell line, while in another aspect, the cells are from primary cell cultures.

In a further aspect, the oncology microarray comprises substantially homogeneous cells expressing a cancer-specific marker. As used herein, “substantially homogeneous” refers to cells which comprise at least about 80% of cells of a given cell type, preferably at least about 90%, at least about 95%, or at least about 100% of cells of a given cell type. Substantially homogeneous cells can be obtained using methods known in the art, such as by flow sorting, panning, magnetic sorting or by using some other affinity-based technique (i.e., relying on antibodies which recognize specific cell types), density gradient centrifugation, cloning (e.g., by limiting dilution), by synchronization), by induction (e.g., by using an agent such as Fas, Apol-2L, by exposing cells expressing a GPCR to its cognate ligand, by exposing cells to chemokines, cytokines, neurotransmitters, adhesion molecules, or by exposing the cells to chemical agents such as forbol esters), and the like. It should be obvious to those of skill in the art that combinations of these methods and other additional methods of sorting cells can be used.

In another aspect, an oncology microarray is provided comprising cells genetically engineered to proliferate abnormally. For example, cells can be genetically engineered to express cell proliferation genes, or to lack tumor suppressor genes, or to express modified forms of such genes. In this aspect, cells may stably or transiently transfected cell lines, or genetically engineered tumors (e.g., such as tumors infected with a recombinant retroviral vector).

Although in one aspect, at least some of the sublocations on the microarrays comprise substantially homogeneous cells, in other aspects, sublocations comprise cells from cancerous tissue which are selected from at least about two of the group consisting of neoplastic cells, fibrous tissue, inflammatory tissue, necrotic cells, apoptotic cells, normal cells, and combinations thereof. In one aspect, each sublocation on the microarray comprising neoplastic cells comprises at least one of: fibrous tissue, inflammatory tissue, necrotic cells and apoptotic cells.

Although in a preferred aspect of the invention, the microarrays comprise human tissues, in one aspect of the invention, abnormally proliferating tissues from other organisms are arrayed. For example, the microarrays can comprise tissues from mice which have either spontaneously developed cancer or which have received transplants of tumor cells. Preferably, the microarray comprises multiple tissues from such mice (e.g., at least about five).

In another aspect of the invention, the microarray comprises tissues from mice which have spontaneously developed cancer or which have received transplants of tumor cells, and which have been treated with a cancer therapy (e.g., drugs, antibodies, protein therapies, gene therapies, antisense therapies, and the like).

In still a further aspect of the invention, tissues from a mouse genetically engineered to over express or under express cell proliferation genes or tumor suppressor genes are provided. In one aspect, a microarray is provided comprising tissues from mice expressing different doses of the same cell proliferation gene or tumor suppressor gene.

Staged Oncology Microarrays

In one aspect of the invention, an oncology microarray is provided comprising a plurality of sublocations which represent different stages of cell proliferation disorder. The microarray can include metastases to tissues other than the primary cancer site. Preferably, the microarray comprises normal tissues from the same patient from whom abnormally proliferating cell and/or tissue samples were derived.

Staging of a Tissue Sample

The inappropriate new growth of cells is neoplasia, and the masses resulting from such inappropriate new growth are termed “neoplasms”. A neoplasm may be either benign or malignant, and the term “cancer” is applied generally to any malignant neoplasm. The term “tumor” originally applied to any neoplasm, benign or malignant. However, in common usage and herein, “tumor” refers to a malignant neoplasm, as does the term “cancer”.

The discrimination between malignant and benign neoplasms is made on the basis of the differentiation status of the neoplastic cells, their rate of growth, local invasion of tissues and metastasis. Benign neoplasms are generally well differentiated, closely resembling the corresponding normal tissue both morphologically and functionally, while malignant tumors may be either well differentiated or poorly differentiated. The lack of differentiation, or anaplasia, that is characteristic of many, but not all, malignant cells is evidenced by changes in the size and shape of the cells themselves, and often by changes in the appearance of the cell nuclei. Anaplastic cells are often larger or smaller than normal cells of the same tissue, and tend to be irregularly shaped. The nuclei of anaplastic cells may also be larger than in normal cells, irregularly shaped, and hyperchromatic.

Benign neoplasms generally have a lower rate of growth than malignant tumors, growing steadily over a period of years or even decades, while malignant tumors tend to grow rapidly and erratically. This generalization is not absolute, however. Some benign tumors can grow more rapidly than some malignant tumors, and benign tumor growth can also be erratic. Generally, growth rate correlates well with differentiation status; poorly differentiated neoplasms tend to have higher growth rates.

Benign neoplasms almost always grow as defined cohesive masses confined to the tissue of origin. A benign neoplasm most often has a layer of connective tissue or capsule largely separating it from surrounding tissues, while malignant tumors tend to lack such defining boundaries.

The most clearly defining difference between benign neoplasms and malignant tumors is the ability of malignant tumors to metastasize or invade tissues other than the tissue of origin. Benign tumors do not have the capacity to metastasize. Invasive tumors have gained the ability to penetrate blood vessels, lymphatic ducts and barriers such as the peritoneum. Tumor cells that have penetrated such a barrier are capable of seeding new tumors at sites distant from the tissue of origin.

One of skill in the art (e.g., a pathologist) can generally determine whether a given neoplasm is benign or malignant by examining the morphology of cells in a tissue sample. It is not possible, however, on the basis of morphology alone, to reliably predict the likelihood that a given tumor will metastasize. Tumors are classified in the art according to grade and stage of the disease, factors that are more useful in predicting disease progression.

Tumor grade is a classification of the degree of differentiation of cells in a tumor. Following diagnosis of cancer on the basis of cell morphology in a biopsy, a tumor is graded with regard to the degree of differentiation in order to begin the process of deciding which treatment options to implement and predicting the outcome of such treatment. The American Joint Commission on Cancer recommends a four tiered tumor grading system, with Grades 1-4. Grade 1 (G1) refers to a well-differentiated tumor, and is often referred to as “low grade”. Grade 2 (G2) refers to a moderately well-differentiated tumor, and is often referred to as “intermediate grade”. Grade 3 (G3) refers to a poorly differentiated tumor which is often referred to as “high grade”, and Grade 4 (G4) refers to an undifferentiated tumor, also referred to as “high grade”. Grades 1 and 2 are generally considered the least aggressive (less likely to invade or metastasize), while grades 3 and 4 are considered the most aggressive.

The classification system recommended by the American Joint Commission on Cancer is widely used by pathologists, but particularly for certain types of cancers, such as soft tissue sarcomas, primary brain tumors, lymphomas, and breast cancer. Other types of cancers are graded using different scales specific to those types of cancer.

The stage of a tumor is determined by considering all available information regarding a patient's tumor relative to what is known about the impact of each particular variable on the patient's prognosis. One accepted staging system is the TNM system (Tumor, Nodes, Metastasis), recommended by the American Joint Commission on Cancer and the International Union Against Cancer. In this system, also referred to as the International Classification System, a number from 0 to 4, describing the tumor's size and spread to adjacent tissues, is assigned for T. A number for N from 0 to 3 is assigned to indicate whether and to what extent the cancer has spread to adjacent lymph nodes, and a number for M (0 or 1) is assigned to indicate the presence of distant metastases. The total TNM score is used to place the tumor into a stage group with a corresponding likely prognosis. For each type of tumor, the higher the stage number, the worse the prognosis. The TNM stage for a given tumor can signify the presence of different groups of indicators, depending on the tissue involved. Examples are given below.

In the TNM system applied to renal cell cancer, for example, stages 1-4 mean the following:

Stage 1: Tumors are less than 2.5 cm, show no evidence of local invasion, no lymph node involvement and no distant metastases.

Stage 2: Tumors are larger than 2.5 cm, show no evidence of local invasion, no lymph node involvement and no distant metastases.

Stage 3: Tumors of any size showing involvement of at least one lymph node, tumors that invade the adrenal gland or surrounding renal tissues, or tumors that invade the renal vein or the inferior vena cava.

Stage 4: Tumors of any size that invade adjacent structures or have evidence of distant metastasis, or any tumor where more than one lymph node is involved.

The standards for determining various TNM stages of cervical carcinoma and endometrial cancer are defined by the Federation Internationale de Gynecologie et d'Obstetrique (FIGO); (Shepherd, 1996, Brit. J. Obst. Gyn. 103: 405-406; Creasman, 1995, Gynecol. Oncol. 58: 157-158). Guidelines for cancer staging for other tumor types are provided by the American Joint Committee on Cancer (AJCC Cancer Staging Manual, 1997, Lippincott-Raven Publishers, 5^{th} Ed., Philadelphia, Pa., pp. 189-194).

Generally, the American Joint Commission on Cancer grading system is useful in the absence of a more specialized grading system for a given type of tumor. Other more specialized grading systems are known to those skilled in the art. The grade of a tumor is but one indicator of prognosis. The process of tumor staging factors the tumor grade for a given tumor along with other prognostic indicia in order to more accurately provide prognostic information. In the methods of the invention, the tumor grade and tumor stage may be used to arrange the samples of a tissue array in order of increasing or decreasing prognosis.

It should be obvious to those of skill in the art that grading systems evolve and that the examples discussed above are non-limiting.

In addition to formal grading schemes, cytogenetic, immunohistochemical and enzymatic function tests may be performed to identify other risk factors or to obtain a molecular profile that is diagnostic and/or prognostic of cancer and/or cancer progression. Taking breast cancer as an example, the expression or altered expression of estrogen receptors, HER2/neu, mutant or wild-type p53, and EGF receptor may be considered in the analysis of tumor prognosis.

Breast Cancer Progression Microarrays

For in situ breast cancers (e.g., ductal carcinoma in situ), grades are assigned on the appearance of their cell nuclei (nuclear grade) and the presence or absence of necrosis. The Van Nuys Prognostic Index considers these two factors along with information regarding the distance of the tumor from the edge of the lumpectomy specimen and the size of the tumor to estimate prognosis (Silverstein & Lagios, 1997, Oncology 11: 393-410).

Breast cancer is graded on the Bloom-Richardson scale, sometimes referred to as the Scharff-Bloom-Richardson scale or Elston-Ellis scale (see Elston & Ellis, 1991, Histopathology 19: 403-410; Frierson et al., 1994, Am. J. Clin. Pathol. 102 (Suppl 1): S3-S8; and Dalton et al., 1994, Cancer 73: 2765-2770). This scheme and those related to it also categorize tumors according to their similarity to the normal tissue architecture. Tubule formation, nuclear pleomorphism and mitotic activity are factored into the scores under this scheme. For invasive cancers, Grade 1 tumors have relatively normal looking cells that are arranged in small tubules. Grade 3 tumors do not have recognizable tubule structures, and Grade 2 tumors are in between (i.e., some tubule structure evident, but poorly organized).

In one aspect, the microarray comprises a plurality of tissues representative of disease progression in breast cancer. Tissues can be selected from the group consisting of normal breast tissue, ductal carcinoma in situ, invasive ductal breast cancer (grade 1), invasive ductal breast cancer (grade 2), invasive ductal breast cancer (grade 3), lymph node metastases from the same any of: ductal carcinoma in situ, invasive ductal breast cancer (grade 1), invasive ductal breast cancer (grade 2), invasive ductal breast cancer (grade 3). In a further aspect of the invention, the at least one control tissue selected from the group comprising of brain, heart, liver, spleen, muscle, lymph node, testis, kidney, thyroid, adrenal gland and prostate, but at least normal breast tissue, is provided on the same or a different microarray. In another aspect of the invention, the sublocations represent Grade I T1N0M0, Grade II T2N1M0, Grade III T3N2M1, Grade IV T3N2M2 tissue samples, Grade HER-2/neu/+, ER/PR+, and Breast ER/PR− (grading according to the World Health Organization).

FIG. 2A shows an example of a breast cancer progression microarray which is part of a profile array substrate. FIG. 2C shows six different tissue specimens representing different stages of breast cancer stained with a CK7 antibody. As shown in FIG. 2D, cancer progression microarrays are preferably provided with along with access to information relating to patients from whom tissue samples on the array were obtained. The information may be written, as shown in FIG. 2D; however, preferably, the microarrays are provided in kits comprising microarray identifiers which can be used to access a specimen-linked database comprising patient information indexed according to the position of a specimen on a particular microarray. A sample of data obtained from a plurality of breast cancer progression arrays is provided in the attached Appendix.

Prostate Cancer Progression Microarray

Prostate cancer typically is classified according to the “Gleason grading system” (see D. F. Gleason, “The Veteran's Administration Cooperative Urologic Research Group: Histologic grading and clinical staging of prostatic carcinoma.” in M. Tannenbaum (ed.) Urologic Pathology: The Prostate. Lea & Febiger, Philadelphia, 1977, pp. 171-198). In the Gleason grading system, there are 5 grades, with grade 5 being the worst with regard to prognosis:

Grade 1 is well-differentiated, closely resembling the normal prostate. Grade 1 prostate cancer are characterized by pale-staining (hematoxylin/eosin stain) glands that grow closely together in a compact mass.

Grade 2 is also well differentiated and has pale-staining glands, but they are more loosely aggregated than Grade 1 cells and tend to invade the surrounding muscle.

Grade 3 is also considered well-differentiated, since it retains a “gland unit” as seen in the normal prostate. The gland unit has a well defined lumen, and each gland unit in Grade 3 tumors is surrounded by prostate muscle, which keeps the gland units separated. However, Grade 3 has extensive invasion of glands into the surrounding muscle. Cells of Grade 3 tumors also stain more darkly and are of variable shapes. Grade 3 is the most common grade seen.

Grade 4 is considered poorly differentiated, and is characterized by the disruption of the normal gland unit. Grade 4 tumors will still have evidence of lumen formation, but the gland units are not clearly distinct, such that the lumens are not separate.

Grade 5 is considered undifferentiated. Grade 5 prostate tumors show no evidence of an attempt to form gland units.

The most important aspect of the Gleason grading system is that it does not stop after the assignment of one grade. In the Gleason system, a pathologist always tries to identify two characteristic patterns and assign a Gleason grade reflecting each one. That is, in a given biopsy, there may be a primary pattern that fits one grade, and a secondary pattern that fits another grade. Rather than setting the grade at the pattern that is most prevalent in a tissue sample, the pathologist assigns grades fitting the two most prevalent patterns. The combination of two grades, known as the “Gleason sum”, was found in Dr. Gleason's original study of 2,900 men to provide a more accurate prognosis estimate than a grade based on a single architectural pattern. In the Gleason system then, the lowest possible score is 2 (only a pattern fitting grade 1 is evident, 1+1=2), and the highest is 10 (only a pattern fitting grade 5 is evident, 5+5=10). Intermediate scores of 7 may result, for example from a sample in which the prevalent pattern fits grade 4 and the minor pattern fits grade 3. Generally, the lower the Gleason score, the better the prognosis for the patient. In addition to the Gleason score, a physician will want to consider other elements in their analysis of prognosis, including, for example, PSA (prostate serum antigen) level and the clinical stage of disease (see below).

Therefore, in one aspect, a microarray is provided which comprises a plurality of cells representative of disease progression in prostate cancer, including sublocations which represent Gleason Grades 1-10, or 1-6, 4-10, and/or 6-10, and including a standard control section, comprising one or more of brain, heart, liver, spleen, muscle, lymph node, testis, kidney, thyroid, adrenal gland and prostate cells/tissue, but at least prostate cells/tissue.

Colon Cancer Progression Microarray

In another aspect, a microarray is provided comprising a plurality of cells representative of disease progression in colorectal cancer. In one aspect, the microarray comprises normal colon mucosa from patients having no history of colorectal cancer and cancerous colon mucosa, preferably from the same patients. Additional samples can include: adenoma with mild dysplasia, adenoma with severe dysplasia, nodal negative colorectal cancer, nodal positive colorectal cancer, paired metastases (e.g., such as lymph node metastases from tumors). Preferably, a standard control section, comprising one or more of brain, heart, liver, spleen, muscle, lymph node, testis, kidney, thyroid, adrenal gland and prostate is provided on the same or a different microarray. In another aspect of the invention, the colon cancer progression microarray comprises at least cells from tumors representing the Colonic 4 Dukes' stages: A, tumor within the intestinal mucosa; B, tumor into muscularis mucosa; C, metastasis to lymph nodes and D, metastasis to other tissues.

Lung Cancer Progression Microarray

In a further aspect, a microarray is provided comprising a plurality of cells representative of disease progression in lung cancer. In one aspect, the microarray comprises normal lung parenchyma, normal bronchi, adenocarcinoma (different subtypes), squamous cell carcinoma, undifferentiated large cell carcinoma, small cell carcinoma, lymph node metastases from tumors included in this microarray (paired metastases). As above, standard control sections, comprising at least one of brain, heart, liver, spleen, muscle, lymph node, testis, kidney, thyroid, adrenal gland and prostate, can be provided on the same or a different microarray.

Sources of Tissue

In one aspect, the cells/tissues at individual sublocations are from cadavers, from autopsies, from surgical specimens, pathology specimens, or represent “clinical waste” that would normally be discarded from other procedures. In addition to tissue sections, microarrays can also include cells from body fluids such as serum, leukapheresis products, pleural effusions, urine (e.g., where the patient has bladder cancer) and the like.

In one aspect of the invention, cell culture lines are used as sources of cancer cells for at least one location. Cell lines can be developed from isolated cancer cells and immortalized with oncogenic viruses (e.g., Epstein Barr Virus).

Exemplary cell lines which can be used in this aspect, include, but are not limited to those listed below:

| | | | Normal Cells from | ATCC |
|---|---|---|---|---|
| Tumor Cell Line | Source | ATCC No. | the Same Patient | No. |
| carcinoma; non-small | lung | CCL-256 | peripheral blood | CCL-256.1 |
| cell lung cancer | | | | |
| adenocarcinoma | lung | CRL-5868 | peripheral blood | CRL-5957 |
| adenocarcinoma | lung | CRL-5872 | peripheral blood | CRL-5958 |
| adenocarcinoma | lung | CRL-5882 | peripheral blood | CRL-5954 |
| adenocarcinoma | lung | CRL-5911 | peripheral blood | CRL-5961 |
| adenocarcinoma | pleural effusion | CRL-5985 | peripheral blood | CRL-5967 |
| adenocarcinoma | lymph node (metastasis) | CRL-5922 | peripheral blood | CRL-5965 |
| carcinoma; classic small | lung | CRL-5886 | peripheral blood | CRL-5959 |
| cell lung cancer | | | | |
| carcinoma; small cell | lung | CRL-5929 | peripheral blood | CRL-5969 |
| lung cancer | | | | |
| carcinoma; small cell | lung | CRL-5931 | peripheral blood | CRL-5956 |
| lung cancer | | | | |
| carcinoma; small cell | lymph node (metastasis) | CRL-5858 | peripheral blood | CRL-5949 |
| lung cancer | | | | |
| carcinoma; small cell | bone marrow | HTB-172 | peripheral blood | CRL-5948 |
| lung cancer | (metastasis) | | | |
| carcinoma; small cell | bone marrow | CRL-5983 | peripheral blood | CRL-5966 |
| lung cancer | (metastasis) | | | |
| carcinoma; small cell | pleural effusion | HTB-120 | peripheral blood | CRL-5947 |
| lung cancer | | | | |
| mesothelioma | pleural effusion | CRL-5915 | peripheral blood | CRL-5963 |
| neuroendocrine | lymph node (metastasis) | CRL-5893 | peripheral blood | CRL-5960 |
| carcinoma | | | | |
| ductal carcinoma | mammary gland; breast | HTB-126 | mammary gland; | HTB-125 |
| | | | breast | |
| ductal carcinoma | mammary gland; breast | CRL-2320 | peripheral blood | CRL-2319 |
| ductal carcinoma | mammary gland; breast | CRL-2338 | peripheral blood | CRL-2339 |
| primary ductal | mammary gland; breast | CRL-2314 | peripheral blood | CRL-2346 |
| carcinoma | | | | |
| primary ductal | mammary gland; breast | CRL-2321 | peripheral blood | CRL-2362 |
| carcinoma | | | | |
| primary ductal | mammary gland; breast | CRL-2322 | peripheral blood | CRL-2323 |
| carcinoma | | | | |
| primary ductal | mammary gland; breast | CRL-2324 | peripheral blood | CRL-2325 |
| carcinoma | | | | |
| primary ductal | mammary gland; breast | CRL-2331 | peripheral blood | CRL-2332 |
| carcinoma | | | | |
| primary ductal | mammary gland; breast | CRL-2336 | peripheral blood | CRL-2337 |
| carcinoma | | | | |
| primary ductal | mammary gland; breast | CRL-2340 | peripheral blood | CRL-2341 |
| carcinoma | | | | |
| primary ductal | mammary gland; breast | CRL-2343 | peripheral blood | CRL-2363 |
| carcinoma | | | | |
| ductal carcinoma | mammary gland; breast | CRL-7345 | skin | CRL-7346 |
| scirrhous | mammary gland; breast | CRL-7482 | skin | CRL-7481 |
| adenocarcinoma | | | | |
| adenocarcinoma | mammary gland; breast | CRL-7484 | skin | CRL-7483 |
| cancer | mammary gland; breast | CRL-7303 | skin | CRL-7302 |
| cancer | mammary gland; breast | CRL-7486 | skin | CRL-7485 |
| carcinoma | mammary gland; breast | CRL-7365 | skin | CRL-7364 |
| carcinoma | mammary gland; breast | CRL-7368 | skin | CRL-7367 |
| carcinoma | mammary gland; breast | CRL-7590 | skin | CRL-7589 |
| malignant melanoma | skin | CRL-1974 | peripheral blood | CRL-1980 |
| basal cell carcinoma | skin | CRL-7762 | skin | CRL-7761 |
| malignant melanoma | skin | CRL-7690 | skin | CRL-7689 |
| melanoma | skin | CRL-7357 | skin | CRL-7356 |
| melanoma | skin | CRL-7360 | skin | CRL-7359 |
| pagetoid sarcoma | skin | CRL-7677 | skin | CRL-7676 |
| benign osteoid osteoma | bone | CRL-7672 | skin | CRL-7671 |
| giant cell sarcoma | bone | CRL-7554 | skin | CRL-7553 |
| heterophilic | bone | CRL-7552 | skin | CRL-7551 |
| osteofication | | | | |
| osteosarcoma | bone | CRL-7444 | skin | CRL-7443 |
| osteosarcoma | bone | CRL-7448 | skin | CRL-7449 |
| osteosarcoma | bone | CRL-7471 | skin | CRL-7865 |
| osteosarcoma | bone | CRL-7496 | skin | CRL-7603 |
| osteosarcoma | bone | CRL-7571 | skin | CRL-7570 |
| osteosarcoma | bone | CRL-7595 | skin | CRL-7519 |
| osteosarcoma | bone | CRL-7622 | lung | CCL-211 |
| osteosarcoma | bone | CRL-7626 | skin | CRL-7625 |
| osteosarcoma | bone | CRL-7628 | skin | CRL-7627 |
| periostitis; granuloma | bone | CRL-7453 | skin | CRL-7452 |
| fibrosarcoma | connective tissue | CRL-7663 | skin | CRL-7662 |
| fibrosarcoma | connective tissue | CRL-7664 | skin | CRL-7662 |
| fibrosarcoma | connective tissue | CRL-7665 | skin | CRL-7662 |
| fibrosarcoma | connective tissue | CRL-7666 | skin | CRL-7662 |
| fibrosarcoma | connective tissue | CRL-7668 | skin | CRL-7662 |
| adenocarcinoma | unknown | CRL-7432 | skin | CRL-7431 |
| transitional cell | ureter | CRL-7886 | skin | CRL-7518 |
| carcinoma | | | | |
| giant cell sarcoma | vertebral column | CRL-7547 | skin | CRL-7546 |

In a further aspect, the cell lines used are primary cell lines, including, but not limited to: colorectal adenocarcinoma (ATCC No. CCL-228); and gastric adenocarcinoma from the stomach (ATCC No. CRL-1864); and melanoma (ATCC No. CRL-1675, CRL-7425). In still a further aspect of the invention, the cell lines are obtained from a metastatic cancer, including but not limited to: lymph node (ATCC No. CCL-227, CRL-7426). Additional cell lines can be obtained through the American Tissue Type Culture collection (www@tcc.org.) which have been developed or characterized at the NCI-Navy Medical Oncology Branch. Cell lines within this collection are catalogued in a database (NCI-Navy Cell Line database) which provides information regarding the patient from whom the cell line is derived (see, e.g. Journal of Cellular Biochemistry Supplement 24: 32-91, 1996, the entirety of which is incorporated by reference herein).

In one aspect, sample tissues are selected from an oncology repository or a collection of tissue specimens that represent the most common neoplastic diseases. These include, but are not limited to:

| Specimen | Disease Type |
|---|---|
| prostate | BPH: adenocarcinoma |
| breast | benign and metastatic, DCIS, stage 1-IV |
| colon | adenoma, adenocarcinoma |
| brain | cerebellar hemangiomas, gliomas |
| ovary | ovarian cancer |
| cervix | cervical cancer, dysplastic, CIN |
| lung | adenocarcinoma, small cell carcinoma |
| hepatic | hepatocellular carcinoma |
| bladder | transitional carcinoma |
| thyroid | medullary carcinoma |
| adrenal | adrenal carcinoma |
| parathyroid | parathyroid tumors |
| pancreas | pancreatic cancer, islet cell tumors |
| soft tissue | Ewing sarcoma |
| integumentary | malignant melanoma, benign nevi |

In one aspect, the microarray comprises at least one sublocation comprising a disease tissue selected from the repository listed above and at least one sublocation comprising a normal tissue, either from the same specimen from which the disease tissue was obtained, or from a normal specimen of the same tissue type (e.g., if the disease tissue is lung, normal lung tissue is selected). In a preferred aspect of the invention, sets of sublocations (e.g., two or more) comprise tissues of the same type but different disease stages.

Tissues are also obtainable from the National Cancer Institute Cooperative Human Tissue Network (http://www.chtn.ims.nci.nih.gov/).

In another aspect, the sublocations are selected from a repository representing cell proliferative disorders affecting women.

| Specimen | Disease Types |
|---|---|
| breast | pagets, cancer, benign disease |
| uterus | endometriosis |
| cervix | dysplasia, HPV, cancer |
| ovaries | diseased, including cancer |
| fallopian tubes | diseased, including cancer |

In one aspect, the microarray comprises at least one sublocation comprising a disease tissue selected from the repository listed above and at least one sublocation comprising a normal tissue, either from the same specimen from which the disease tissue obtained, or from a normal specimen of the same tissue type from which the disease tissue is obtained. In another aspect of the invention, sets of sublocations (e.g., two or more) comprise tissues of the same type but representing different disease stages.

In another aspect of the invention, sublocations are selected from a repository of endocrine tissue specimens from patients having cell proliferative disorders:

| Specimen | Disease Types |
|---|---|
| thyroid | hyper and hypo thyroidism |
| parathyroid | cancer, adenoma |
| adrenals | adenoma, cancer |
| pancreas | diabetes, islet cell tumors |
| breast | hyperplasia, tumor |
| ovary | benign cancer |
| bone | estrogen replacement, osteoporosis |
| prostate | bph, cancer |

In one aspect, the microarray comprises at least one sublocation comprising a disease tissue selected from the endocrine tissue repository listed above and at least one sublocation comprising a normal tissue, either from the same specimen from which the disease tissue was obtained, or from a normal specimen of the same tissue type. In another aspect of the invention, sets of sublocations (e.g., two or more) comprise tissues of the same type but representing different disease stages.

As discussed above, normal cell/tissue samples can be provided on the same or different microarrays as those comprising abnormally proliferating cells. In one aspect of the invention, normal cells/tissues are selected from the group consisting of cerebrum, cerebellum, heart, lung, thyroid gland, adrenal gland, skin, parotis, pancreas, stomach (corpus), stomach (antrum), small intestine, colon, liver, gall bladder, tonsil, spleen, lymph node, endometrium, (proliferation), endometrium(secretion), placenta (last trimenon), placenta (first trimenon), kidney, prostate, testis, epidydimis, skeletal muscle, smooth muscle. Preferably, such an array comprises duplicate samples of a given cell/tissue type.

Construction of Microarrays

Preparing Donor Cell/Tissue Blocks

In one aspect of the invention, cells and/or tissues are obtained and either paraffin-embedded, plastic-embedded or frozen into blocks from which portions (donor samples) can be obtained. Frozen tissues preferably are obtained where non-fixed samples are desired (e.g., when detecting nucleic acids). When paraffin- or plastic-embedded, a variety of tissue fixation techniques may be used to preserve the morphology of cellular structures within the samples. Examples of fixatives, include, but are not limited to, aldehyde fixatives such as formaldehyde; formalin or formol; glyoxal; glutaraldehyde; hydroxyadipaldehyde; crotonaldehyde; methacrolein; acetaldehyde; malonaldehyde; malialdehyde; and succinaldehyde; chloral hydrate; diethylpyrocarbonate; alcohols such as methanol and ethanol; acetone; lead fixatives such as basic lead acetates and lead citrate; mercuric salts such as mercuric chloride; formaldehyde; dichromate fluids; chromates; picric acid, and heat. Tissues are fixed until they are sufficiently hard to embed. The type of fixative employed will be determined by the type of molecular procedure being used, e.g., where the molecular characteristic(s) being examined include the expression of nucleic acids, isopentane, or PVA, or another alcohol-based fixative is preferred.

Embedding medium encompassed within the scope of the invention, includes, but is not limited to, paraffin or other waxes, plastic, gelatin, agar, polyethlene glycols, polyvinyl alcohol, celloidin, nitrocelluloses, methyl and butyl methacrylate resins or epoxy resins. Water-insoluble embedding media such as paraffin and nitrocellulose require that specimens be dehydrated in several changes of solvent such as ethyl alcohol, acetone, xylene, toluene, benzene, petroleum, ether, chloroform, carbon tetrachloride, carbon bisulfide, and cedar oil. or isopropyl alcohol prior to immersion in a solvent in which the embedding medium is soluble. Water soluble embedding media such as polyvinyl alcohol, carbowax (polyethylene glycols), gelatin, and agar, can also be used.

In one aspect, specimens are freeze-dried by deep freezing in plastic cassettes and storing them at −80-70° C., such as in liquid nitrogen, forming frozen donor blocks. Preferably, the samples are then covered with a cryogenic media, such as OCT®, and kept at −80-70° C., until sectioned. Examples of embedding media for frozen samples include, but are not limited to, OCT, Histoprep®, TBS, CRYO-Gel®, and gelatin, to name a few. In another aspect, a freezing aerosol may be used to facilitate embedding of the donor sample block. An example of a freezing aerosol is tetrafluoroethane 2.2.

Cell donor blocks (comprising one or more cells typically found non-adherent in an organism and/or comprising cells which have been purified to be substantially homogeneous) are generated by washing cells one or more times in a suitable buffer which does not lyse the cells. The cells are collected by centrifugation and resuspended in a fixative and after fixation are centrifuged again and resuspended in an embedding material, such as plastic or paraffin. Alternatively, where cells are resuspended in a fast-freezing embedding material such as OCT, no prior fixation step is needed. Preferably, cells in embedding material are transferred to a mold which as a support web or plastic block. The cells and embedding material also can be co-centrifuged prior to being enclosed. The generation of cell blocks is described in EP 408,225; U.S. Pat. No. 4,822,495; U.S. Pat. No. 5,137,710; U.S. Pat. No. 5,817,032; and U.S. Pat. No. 4,656,047, the entireties of which are incorporated by reference herein.

Other methods known in the art may also be used to facilitate embedding of a cell or tissue sample to form a donor block.

Preparing Microarray Blocks

Blocks for receiving donor cell/tissue samples or “recipient blocks” generally are formed by providing a suitable embedding material and coring one or more holes into the material after it has hardened. The holes are sized to receive a desired cell or tissue donor sample from the donor block and when holes in the recipient block are filled with a desired number of donor samples, a microarray block is formed.

Information regarding the coordinates of the hole and the identity of the tissue sample at that hole is recorded, effectively addressing each sublocation on microarrays generated by sectioning the microarray block and placing each section on a substrate (e.g., such as a glass slide).

In a preferred aspect of the present invention, the donor sample is obtained by boring an elongated sample core from the donor block and placing the core in a hole cored in a recipient block at substantially the same time.

In another aspect, the recipient block is prepared prior to obtaining specimens from donor block(s). For example, the recipient block can be prepared by placing a fast-freezing, cryo-embedding matrix in a container and freezing the matrix so as to create a solid, -frozen block. Freezing can be facilitated using tetrafluorethane 2.2, or by any other methods known in the art. The block can be cored as described above, in preparation for receiving samples, and stored until needed to generate a microarray block.

Holes in the recipient block can be of any shape and size, but preferably are made in a regular pattern. In one aspect of the invention, the holes are elongated in shape. In another aspect, the holes are cylindrical in shape. Preferably, holes are sized to receive a cylindrical donor sample of about 1-4 mm long with a diameter of about 0.1-4 mm, and preferably, from about 0.3-2.0 mm. More preferably, the cylinder diameter is less than about 1.0 mm, for example, about 0.6 mm.

The coring process may be automated using core needles coupled to a motor or some other source of electrical or mechanical power. In one aspect of the invention, a microarray block is generated using a Beecher Instruments Tissue Arrayer (Beecher Instruments, Silver Springs, Md.). This device basically consists of a turret containing two hollow core borer needles mounted on a platform with a spring mechanism. A smaller needle removes a core from the recipient block while a larger needle removes a core of tissue from the donor tissue block and by means of a stylet. The stylet is inserted into the smaller needle thereby injecting the donor tissue core into the hole made in the recipient block.

In one aspect of the invention, the recipient block is a paraffin block and the donor block(s) are individual archival tissue blocks that have been assembled for a particular type of microarray. In this aspect, an empty recipient block is placed in a holder and is held in position by restraining element, for example, magnets built into the arrayer. In one aspect, the arraying process is started by setting a spacing mechanism for controlling the spacing between cores/sublocations and X-Y coordinates, to zero. In one aspect, the arrayer comprises a first hollow needle and a second hollow needle, the first needle smaller being smaller than the second needle. A depth stop which controls the depth of the hole created by the first needle is set to a selected depth (e.g., 0.5-1.0 mm) and the smaller needle is pushed downward (e.g., by hand). When the depth stop blocks the downward motion of the needle, the needle is rotated (e.g., 45 degrees), facilitating the retrieval of a paraffin core whose diameter is defined by the bore of the first needle from the block. Downward pressure is now released and the needle, by spring action, or through some other mechanical or electrical force, moves upward. A stylet is used to empty the first needle and the paraffin core is discarded, leaving a recipient block comprising at least one hollow core for receiving a tissue or cell sample.

In one aspect, after a hole is made by the needle, a holder for a block of embedded donor tissue (e.g., frozen tissue or paraffin-embedded tissue) is placed over the recipient block with the core, and the donor block is placed on top or the holder. In one aspect, the holder is in the form of a block bridge or table. The needle turret is rotated and a second needle, which is larger than the first needle in diameter, is placed in position. In one aspect, the second needle has an inside diameter of approximately 600 μm.

In other aspects of the invention, particularly where it is desired to obtain frozen microarray blocks, a microarrayer such as the one described in U.S. patent application Ser. No. 09/779,753 filed Feb. 8, 2001, is used. The entirety of this application is incorporated herein by reference.

Preferably, desired coordinates of the donor block to obtain a sample from are identified prior to coring a donor sample. For example, a section of the donor block can be treated to make tissue/cell morphology visible under a microscope (e.g., by hematoxylin and eosin staining) and a control sampling area can be identified and marked on the donor block. A donor core sample is then obtained from the sampling area.

In still other aspects, desired coordinates can be identified by reacting sections with one or more molecular probes and identifying coordinates in the donor block comprising cell(s) which do or do not express a particular biological characteristic of interest identified by the reaction or lack of reaction of an area of the section with the probe.

Generally, the order of donor samples within a recipient block can be varied to suit a user's needs. In a preferred aspect, microarrays comprise a plurality of tumor samples and different grades or stages of each tumor are represented on the array. Preferably, normal cell and/or tissue samples are provided in the recipient block as well. Still more preferably, samples represent the progression of cancer from its earliest stage to its most advanced. Samples can also be arranged according to treatment approach, treatment outcome or prognosis, or according to any other scheme that facilitates the subsequent analysis of the samples and the data associated with them.

The finished recipient block, now a microarray block, comprising at least two cores of cell samples and/or tissue samples from the same or different donor blocks is then sectioned to about 2 μm-20 μm with a microtome or other cutting implement. Sections preferably are mounted and on a substrate to facilitate handling and/or storage. In one aspect, the microarray block is sectioned at about 4-10 μm and the substrate is an about 1 inch×3 inch positively charged microscope slide.

Other methods of generating microarrays are described in U. S. Provisional Application No. 60/213,321, the entirety of which is incorporated by reference herein, and in WO 99/44062 and WO 99/44062, incorporated entirely by reference herein, and are encompassed within the scope of the instant invention.

Preparation of Large Format Frozen Tissue Arrays

In one aspect of the invention, frozen microarrays are provided in which at least one sublocation comprises at least about two different types of cells. Preferably, the at least one sublocation comprises a section through a donor core sample having a diameter of larger than about 0.6 mm. Samples for such “large format” microarrays can include samples from repositories comprising frozen cells and/or tissue stored at about −80° C.-20° C.

In one aspect, about 20 samples of tumor specimens are obtained for one array and 20 samples of normal tissues are obtained for a second array. A portion measuring approximately 2×2×2 mm is taken from each of the collected tissue specimens and smaller portions of tissue are arranged on a 2 mm thick layer of frozen cryogenic embedding compound (e.g., OCT) that has been previously set into a plastic embedding mold that measures 37×24×5 mm and frozen in a cryostat, thereby forming an “array block”. The location of each specimen as it is placed in the array block is noted so that the identity of each specimen is maintained with 100% accuracy. After each specimen is set into the array block and its location is noted, the embedding mold containing the array block is then filled with additional OCT compound and allowed to completely freeze.

Once frozen, the block of OCT compound containing the tissue array is removed from the mold and mounted on a cryostat chuck using additional OCT compound as an adhesive. The chuck is allowed to freeze onto the array block to ensure a firm bond. When frozen, the chuck is placed on a microtome within a cryostat and sectioned in the same manner as a routine frozen section at about 4-6 μm. Sections are then mounted on a positively-charged substrate, such as a glass microscope slide. The substrate can then be stained using any method that can be performed on frozen sections, such as methods which employ routine and special stains, as well as immunohistochemistry and in situ hybridization. The block can be stored for a period of time in a −80° C. freezer for future use.

As above, data relating to the expression of one or more biological characteristics of samples of the large format array are preferably recorded, indexed according to the location of the samples of the array. Preferably, this information includes patient information.

It should be obvious to those of ordinary skill in the art that although dimensions of elements used in the above procedure can vary and that such variations are encompassed within the scope of the invention. The large formats arrays according to the present invention are particularly useful for arraying samples of cancerous tissue which include a plurality of different cell types, such as one or more of: stromal cells, extracellular matrix, necrotic, cells, and apoptotic cells, in addition to abnormally proliferating cells. Large format arrays can be used alone or in conjunction with small format arrays (e.g., comprising samples of one cell type or of about 0.6 mm or less in diameter). In one aspect of the invention, a large format array is used in conjunction with a small format array derived from the same patient's tissue sample. In this aspect, the large format array can be used to demonstrate that the biological characteristics of the smaller sublocations of a small format array are representative of the biological characteristics within a larger sample of tissue.

Mixed Format Microarrays

In another aspect of the invention, microarrays are generated using heterogeneous samples, e.g., such as paraffin-embedded and/or plastic-embedded, and frozen samples, all provided in the same microarray block. In still another aspect, at least one sublocation of the microarray comprises cells from a serum sample or other sample of body fluid (e.g., blood, urine, CSF, a perfusion sample, and the like). Preferably, both cells and tissue samples from the same patient are provided in a single microarray block. Still more preferably, a microarray block is provided comprising samples representing at least about five different tissue types from the same patient. This optimizes the simultaneous use of the microarrays to examine cell and/or tissue morphology alongside with nucleic acid and protein expression by providing tissues in formats which are especially suited for particular assays. For example, while morphology will be maximized in paraffin-embedded or plastic-embedded sections, nucleic acid detection will be maximized using frozen sections or frozen samples from a bodily fluid.

In a preferred aspect, a microarray is used to screen for cancer-specific markers which are both diagnostic of disease progression and which can be assayed for in a bodily fluid. Such markers are particularly amenable for diagnostic/prognostic tests in clinical settings since they can be obtained readily from patients with minimally invasive measures.

Multiple donor blocks can be used to provide cores of samples which are either paraffin-embedded, or plastic-embedded, or frozen. Preferably, the recipient block/microarray block comprises a fast-freezing embedding material. More preferably, microarrayers such as those described in U.S. patent application Ser. No. 09/779,753 are used to create mixed format microarray blocks according to the invention.

Methods of Using Oncology Microarrays

The oncology microarrays according to the invention allow a user to access large data sets, to establish molecular profiles relating biological characteristics to the progression, recurrence, and response to treatment of neoplastic tissues, and to discover diagnostic/prognostic correlations relating biological characteristics with phenotypes. Each microarray arrays a plurality of different types of biomolecules on a single substrate. One to thousands of genes or proteins can be analyzed from the same set of clinical samples and a database can be constructed relating alterations in the expression and/or form of one or more biomolecules to the occurrence, progression and/or recurrence of cancer.

Applications for the oncology tissue microarrays according to the invention include, but are not limited to:

selecting promising gene targets

sorting/prioritizing cDNA array data

surveying entire populations

validating gene discoveries in 100's of human tissue specimens

investigating pathogenesis and progression in cell proliferative disorders

searching for diagnostic, prognostic and clinical correlations

performing comprehensive molecular profiling of large numbers of specimens

For Prognostic/Predictive Indication-Immunohistochemistry (IHC): Automated and Manual Methods

Most treatments for breast cancer are based on prognostic and predictive factors of which the traditional staging variables (tumor size, node status, metastasis) being the most important. Estrogen (ER) and Progesterone receptors (PgR) status, as determined by IHC, are the only two predictive factors recommended for clinical use. However, many other antibodies such as Ki67, c-erbB-2 and p53 are also being studied by IHC for their prognostic value but appear to be more a valuable in the predictive sense.

In one aspect, substrates comprising breast cancer progression microarrays as described above provide sublocations which have already been identified as being positive or negative for a particular biomarker (such as a marker specifically recognized by an antibody or nucleic acid probe). Up to at least about 20 different types of breast cancers (from 20 individuals) and normal breast samples can be tested simultaneously with a test tissue to compare staining variability and antibody sensitivity on a single substrate at a single time. This provides a more accurate interpretation of results along with the quality assurance of laboratory competence.

In one aspect, the test sample is obtained from an individual suspected of having a disease (e.g., cancer), and is placed on a profile array substrate at a first location, the profile array substrate comprising at a second location, a microarray comprising a plurality of sublocations which each represent different stages in the progression of a disease. The test sample and the microarray are contacted with a molecular probe reactive with a biomolecule (e.g., an antibody specific for a tumor specific antigen, a nucleic acid probe which specifically hybridizes to another nucleic acid, an enzyme capable of recognizing a substrate bound to an antibody, polypeptide, nucleic acid), and the reactivity of the molecular probe is measured to provide an indicia of the presence or absence of the biomolecule. Reactivity can be any of binding, cleavage, processing, and/or labeling, and the like. Reactivity of the molecular probe with the test sample is compared with reactivity of the molecular probe with the different sublocations on the microarray. In one aspect of the invention, reactivity of the sublocations on the microarray in at least one test sample is known and is characteristic of a biological trait, such that reactivity of the test sample is indicative that the test sample shares that biological trait.

Preferably, data relating to the reactivity of the test sample and the sublocations of the microarray is entered into a specimen-linked database, and information relating to the expression of the biological trait in different samples of the microarray is made accessible, along with other data relating to the samples (e.g., such as patient information) to the user. Preferably, the database represents information from a population of individuals. In one aspect of the invention, the individual from whom a test sample is obtained has at least one trait in common with the population.

In a particularly preferred aspect of the invention, data relating to an image of the test sample is stored within the database, and the image can be displayed by the user upon accessing the database.

In one aspect of the invention, reactivity of the molecular probe with different cell and/or tissue samples on the microarray is not known, and information relating to reactivity with the test sample and the cells in different sublocations in the array is determined and entered into a database. In another aspect of the invention, the test sample is contacted with different distinguishable molecular probes (e.g., a fluorescent antibody specific for Her-2/neu and a rhodamine labeled antibody specific for PSA), and a plurality of different reactivities is determined, and entered into the database. In still another aspect of the invention, sets of substantially identical microarrays (e.g., from the same recipient block) are assayed in parallel using multiple samples of the same test tissue (e.g., from neighboring sections of a test block of embedded test tissue), expanding the number of different molecular probes being tested against the test sample. In this way, a molecular profile of the test sample can be determined and compared with the molecular profile of the set of microarray samples. Most preferably, both RNA transcript molecular profiles and protein molecular profiles are obtained from identical or substantially identical sets of microarrays.

In one aspect of the invention, relationships are identified between the biological characteristics of a test sample and cells/tissues on the microarray, or from other previously characterized cells/tissues, by using a processor which accesses a database of information relating to the previously characterized tissues or cells and/or the patients from whom these cells and/or tissues were obtained. Programs for identifying relationships between data sets are known in the art and include the Spotfire™ program as described in U.S. Pat. No. 6,014,661, the entirety of which is incorporated by reference herein.

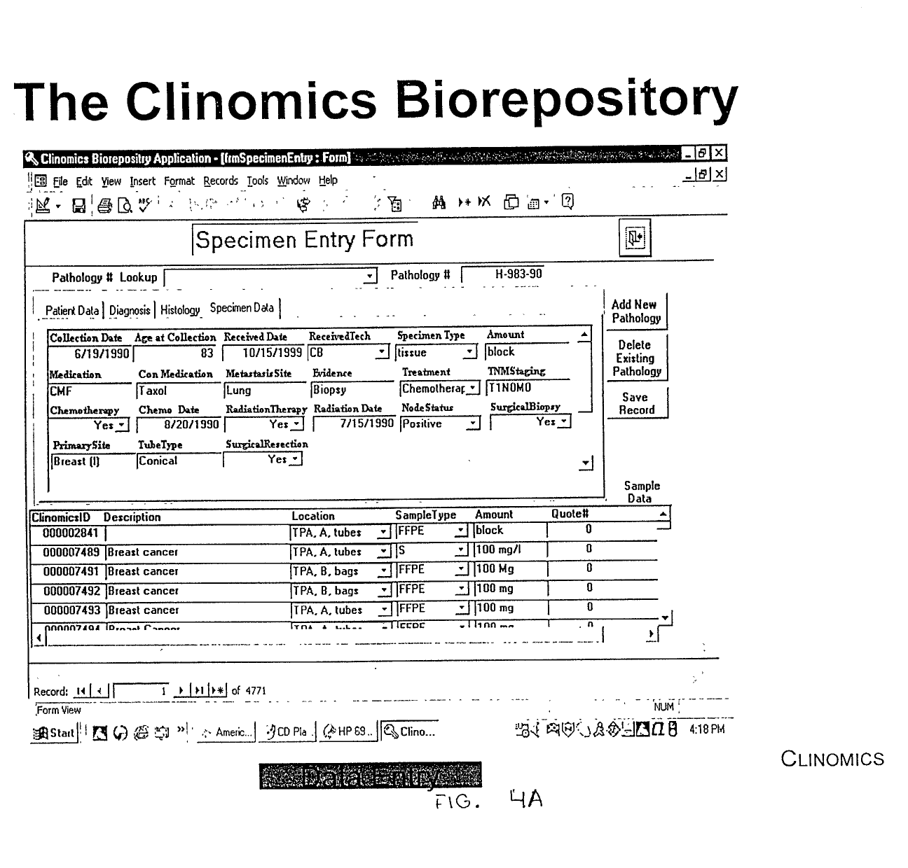

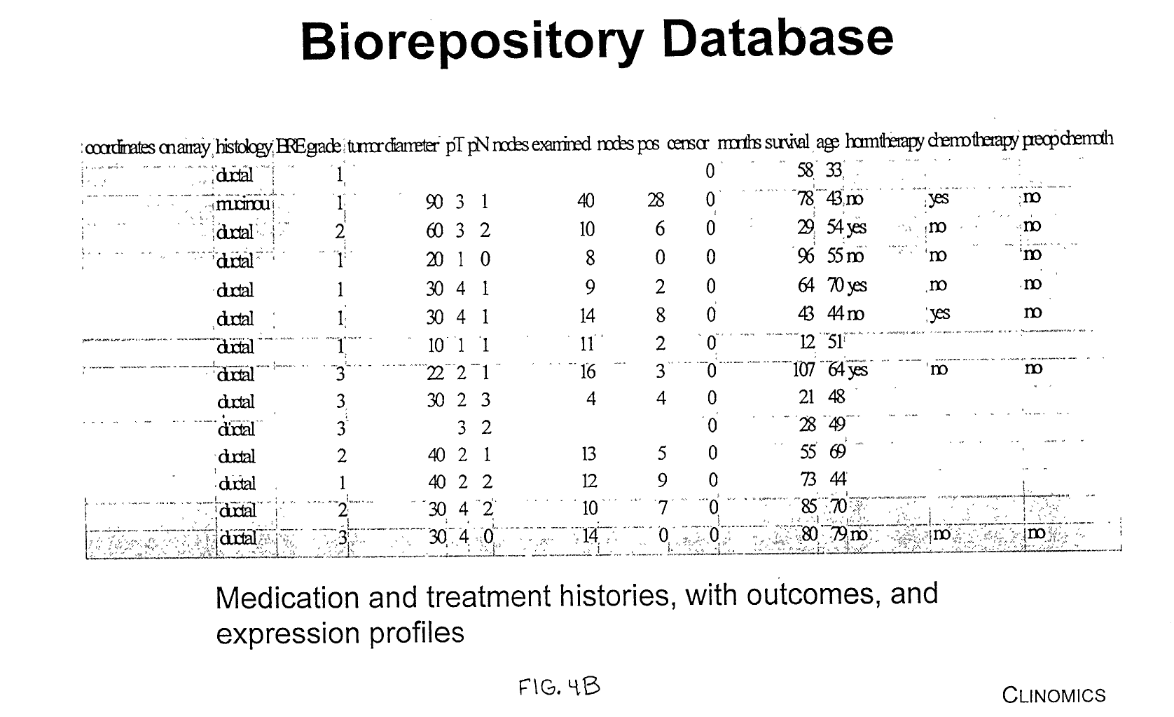

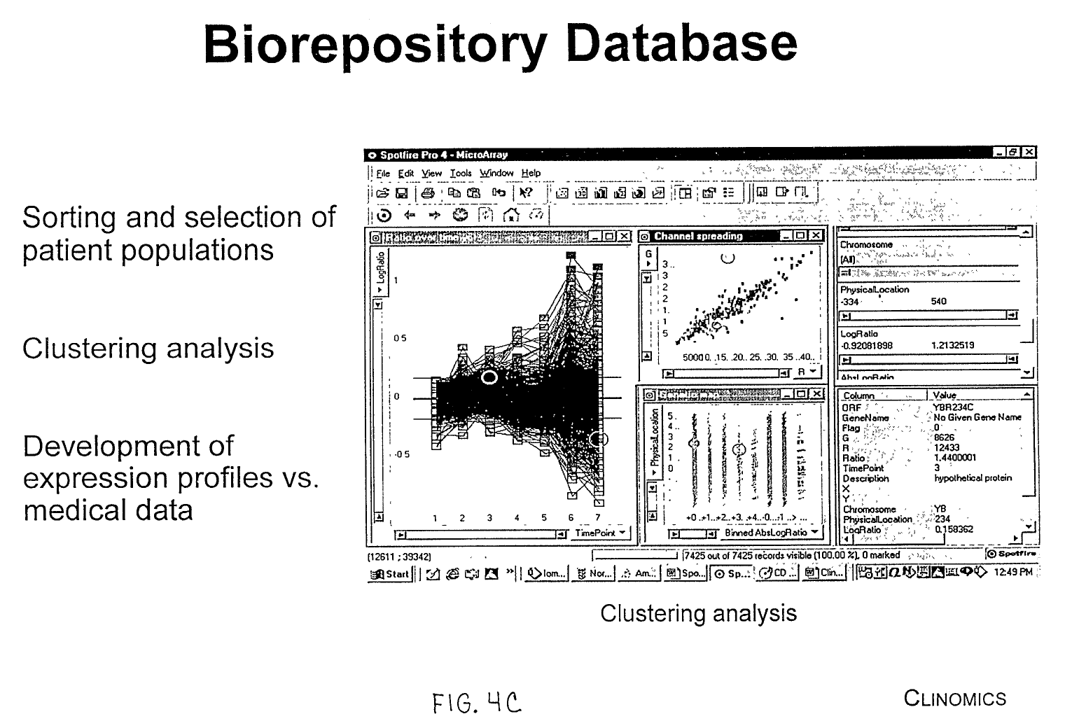

In a preferred aspect, a user of a microarray is provided with access to information regarding the microarray. In one aspect, as shown in FIG. 2D, this information is in the form of printed information regarding the cells/tissues on the microarray. In another aspect of the invention, the user is provided with access to a processor (e.g., a device connectable to a network). The processor communicates with a database (either stored within the memory of the processor or in a server which the processor accesses through a client). In one aspect, the processor downloads records relating to the particular tissues on the microarray and classifies them by type or attribute (e.g., patient sex, age, disease, exposure to drug, tissue type, cancer grade, and the like) (see, e.g., FIGS. 4A-4C). Access to the database can be provided by providing a user with an identifier of the microarray which the user can input into the display of a user device connectable to the network. Upon receiving the input, the user device displays an interface which comprises information relating to the microarray and/or links to portions of the database comprising information relating to different samples on the microarray.

The processor analyzes the relationships between the stored data and the data relating to the test tissue using any method standardly used in the art, including, but not limited to, regression, decision trees, neural networks, and fuzzy logic, and combinations thereof. The processor displays at least one relationship or identifies that no discernable relationship can be found. In one aspect, the processor displays a plurality of relationships on the interface of a display (e.g., on a computer or a wireless device connectable to a network) and displays information relating to the statistical probability that the relationship exists (e.g., whether or not a correlation can be found). The user selects among a plurality of relationships identified by the processor by interfacing with the interface to determine those of interest (e.g., a relationship which is a disease might be of interest while a relationship regarding hair color might not be). In one aspect of the invention, rather than scanning an entire database, the system samples the database randomly until at least one statistically satisfactory relationship is identified, with the user setting parameters for what is “statistically satisfactory.”

In one aspect of the invention, the relationship of interest is used to provide a diagnosis of a disease. In another aspect of the invention, the relationship of interest is used to identify the biological role of an uncharacterized gene. In another aspect of the invention, the processor accesses other databases which comprise information relating to medical treatment of a particular disease, for example, demographic information, or actuarial data, relating to individuals who are the source of the tissue, and other information to further define relationships between the biological characteristics of the test tissue and the tissues for which information exists in the database.

Use of Cancer-Specific Markers To Evaluate Cancer Progression in Oncology Microarrays

In one aspect of the invention, the biological characteristic being assayed is the expression or form of a cancer-specific marker. As used herein, “a cancer-specific marker” or a “tumor specific antigen” is a biomolecule which is expressed preferentially on cancer cells and is not expressed or is expressed to small degree in noncancer cells of an adult individual. As used herein, “a small degree” means that the difference in expression of the marker in cancer cells and noncancer cells is large enough to be detected as a statistically significant difference when using routine statistical methods to within 95% confidence levels. A cancer-specific marker is any biomolecule that is involved in or correlates with the pathogenesis of a cell proliferative disease, and can act in a positive or negative manner, as long some aspect of its expression or form influences or correlates with the presence or progression of a cell proliferative disease. While in one aspect, expressed levels of a biomolecule provide an indicia of cancer progression or reoccurrence, in another aspect of the invention, the expressed form of a biomolecule provides the indicia (e.g., a cleaved or uncleaved state, a phosphorylated or unphosphorylated state).

In one aspect of the invention, the expression characteristics of cancer-specific markers are determined in test samples and compared to the expression characteristics of the marker in any of the oncology arrays described above. In one aspect, the cancer-specific marker is the product of a characterized gene. In another aspect, a cancer-specific marker is a cell growth related polypeptide which promotes cell proliferation. In a preferred aspect, the expression of the cancer specific marker is used to prognose and/or predict reoccurrence of abnormal cell proliferation.

Non-limiting examples of cancer-specific markers include growth factors, growth factor receptors, signal transduction pathway participants, and transcription factors involved in activating genes necessary for cell proliferation. Alternatively, or in addition, cell proliferative genes may function to suppress cell proliferation. Non-limiting examples include tumor suppressor genes (e.g., p57kip2, p53, Rb) and growth factors that act in a negative manner (e.g., TGF-β). A loss or alteration in the function of a negatively acting growth regulator often has a positive effect on cell proliferation.

Among the cell growth related polypeptides, the cyclin-dependent kinase inhibitor p57Kip2 is of interest for its apparent tumor suppressor activity. The gene encoding the human p57Kip2 was located at 11p15.5 (Matsuoka et al., 1995, Genes Dev. 9: 650-662). This chromosomal region is known to develop frequent loss of heterozygosity implicated in a number of human cancers, including Wilms' Tumor, and Beckwith-Wiedemann syndrome (BWS), a cancer syndrome. BWS is characterized by numerous growth abnormalities and an increased risk of childhood tumors.

Several types of childhood tumors, including Wilms' tumor, adrenocortical carcinoma and rhabdomyosarcoma display a specific loss of maternal 11p15 alleles, suggesting that genomic imprinting at that locus plays an important role in the function of genes at that locus. This region also contains imprinted genes encoding Insulin-like Growth Factor II (IGF-II) and H19, both of which are implicated in adrenal neoplasms. The p57Kip2 polypeptide, also known as CDKN1C, is a potent tight-binding inhibitor of several kinases instrumental in the regulation of the G1 phase of the cell cycle. p57Kip2 negatively regulates cell proliferation. The growth inhibitory action of p57Kip2 and its association with a genomic locus showing frequent loss of heterozygosity highlight p57Kip2 as a candidate tumor suppressor.

Transcription of the p57kip2 gene results in the generation of a major transcript of 1.5 kb and a minor transcript of 7 kb. Compared with the related CDK inhibitors p21Waf1 and p27Kip1, the tissue distribution of p57Kip2 expression is limited (Lee et al., 1995, Genes Dev. 9: 639-49). The major 1.5 kb transcript is expressed at high levels in placenta, and at relatively lower levels in muscle, kidney, pancreas and heart. The 7 kb mRNA is also expressed in skeletal muscle and the heart (Lee et al., supra). The p57kip2 gene is also strongly expressed in the prostate.

The p57Kip2 polypeptide is a 348 amino acid protein with a calculated molecular mass of 37.3 kD. The polypeptide migrates anomalously on SDS PAGE, with an apparent relative molecular weight of 57 kD. The polypeptide comprises four primary domains. N-terminal amino acids 30 to 86 comprise a p21/27-related CDK inhibitory domain. After the inhibitory domain is a proline-rich domain comprising a MAP kinase consensus phosphorylation site. This is followed by an acidic domain from residues 178 to 284. Finally, the C-terminus of the polypeptide has sequence conservation with the C-terminus of p27Kip1 and includes a nuclear localization signal and a CDK consensus phosphorylation site (Matsuoka et al., 1995, supra).

Overexpression of p57Kip2 arrests cells in G1. p57Kip2 can bind CDK2, CDK3, CDK4 and cyclins E, A and D1 and is able to inhibit the H1 kinase activity of cyclin E-CDK2 and cyclin A-CDK2. Inhibition is more potent against G1 CDK than against the mitotic CDK cyclin B1-CDK1. p57Kip2 can bind to cyclin/CDK complexes in a cyclin dependent manner (Matsuoka et al. 1995, supra).

Immunohistochemical studies have shown that p57Kip2 is localized to the nucleus in normal tissues. p57Kip2 is expressed in terminally differentiated cells, suggesting an involvement of this CKI in cell cycle exit during differentiation (Matsuoka et al., 1995, supra).

The so-called tumor antigens are also included among the growth-related polypeptides. Tumor antigens are a class of protein markers that tend to be expressed to a greater extent by transformed tumor cells than by non-transformed cells. As such, tumor antigens may be expressed by non-tumor cells, although usually at lower concentrations or during an earlier developmental stage of a tissue or organism. Tumor antigens include, but are not limited to, prostate specific antigen (PSA; Osterling, 1991, J. Urol., 145; 907-923), epithelial membrane antigen (multiple epithelial carcinomas; Pinkus et al., 1986, Am. J. Clin. Pathol. 85: 269-277), CYFRA 21-1 (lung cancer; Lai et al., 1999, Jpn. J. Clin. Oncol. 29: 421-421) and Ep-CAM (pan-carcinoma; Chaubal et al., 1999, Anticancer Res. 19: 2237-2242). Additional examples of tumor antigens include CA125 (ovarian cancer), intact monoclonal immunoglobulin or light chain fragments (myeloma), and the beta subunit of human chorionic gonadotropin (HCG, germ cell tumors).

A sub-category of tumor antigens includes the oncofetal tumor antigens. The oncofetal tumor antigens alphafetoprotein and carcinoembryonic antigen (CEA) are usually only highly expressed in developing embryos, but are frequently highly expressed by tumors of the liver and colon, respectively, in adults. Other oncofetal tumor antigens include, but are not limited to, placental alkaline phosphatase (Deonarain et al., 1997, Protein Eng. 10: 89-98; Travers & Bodmer, 1984, Int. J. Cancer 33: 633-641), sialyl-Lewis X (adenocarcinoma, Wittig et al., 1996, Int. J. Cancer 67: 80-85), CA-125 and CA-19 (gastrointestinal , hepatic, and gynecological tumors; Pitkanen et al., 1994, Pediatr. Res. 35: 205-208), TAG-72 (colorectal tumors; Gaudagni et al., 1996, Anticancer Res. 16: 2141-2148), epithelial glycoprotein 2 (pan-carcinoma expression; Roovers et al., 1998, Br. J. Cancer. 78: 1407-1416), pancreatic oncofetal antigen (Kithier et al., 1992, Tumor Biol. 13: 343-351), 5T4 (gastric carcinoma; Starzynska et al., 1998, Eur. J. Gastroenterol. Hepatol. 10: 479-484,; alphafetoprotein receptor (multiple tumor types, particularly mammary tumors; Moro et al., 1993, Tumour Biol. 14: 11-130), and M2A (germ cell neoplasia; Marks et al., 1999, Brit. J. Cancer 80: 569-578).

The expression characteristics of cell growth related polypeptides are critical not only to their function, but also to their usefulness as prognostic or diagnostic indicators of disease. For example, when a given polypeptide (e.g., a tumor-suppressor gene product) or the RNA encoding it is used as a diagnostic or prognostic indicator, there are several characteristics of its expression that may be relevant. First, the total level of expression in the tumor, relative to the expression in normal cells of the corresponding cell type is important. In one aspect of the invention, the total level of expression is determined by, for example, immunoblot analysis or Northern Blot analysis. For a tumor suppressor gene, for example, a lower level of the tumor suppressor gene product in tumor samples suggests that the lack of the tumor suppressor protein may be involved in the progression of the tumor.

Even when no definitive mechanism of action in tumor etiology is known, the correlation of any expression characteristic (e.g., higher or lower expression) of a given polypeptide or its RNA with a particular clinical diagnosis or outcome in other patients makes the expression characteristics of that polypeptide or its RNA useful in the diagnosis or prognosis of disease. The level of expression of the given polypeptide or its RNA in a particular patient is used, along with the known correlation with its expression in that disease, to diagnose or predict a clinical outcome for that patient.

Another expression characteristic is the percentage of cells expressing the polypeptide in a given tissue sample. It is often found that not all cells of a given tumor express the same markers. Further, within the population of cells that do express a polypeptide, the extent of that expression can vary from cell to cell. In one aspect, the percentage of cells expressing the polypeptide is the criterion used in diagnosis and prognosis. In this aspect, the extent to which positive cells express the polypeptide is a diagnostic characteristic. For immunohistochemical detection, for example, the percentage of positive-staining cells can be further divided into those cells that express the polypeptide to a high, medium, or relatively low level (obviously, any other subdivision scheme may be used). It is possible, for example, that high expression by relatively few cells correlates with a different prognosis than low expression by a larger number of cells, even though the total expression level between two such samples is approximately the same.

Another expression characteristic that can be useful is the localization of expression of the polypeptide. If, for example, the growth-related polypeptide is only expressed in certain cells of a functional structure within a tissue, a change of the expression within those cells that is found to correlate with a disease or the prognosis of that disease can be a useful characteristic. For instance, a marker that is normally expressed only in cells lining the lumen of a glandular structure may become more widely expressed throughout a tissue as the tissue becomes transformed.

In addition to localization within a tissue, the cellular localization of a polypeptide can be an important expression characteristic in disease prognosis or diagnosis. If, for example, a polypeptide that is normally predominantly cytoplasmic becomes predominantly nuclear in a disease, that change can be useful as a diagnostic or prognostic indicator.

Another expression characteristic that can be useful is a change in the conformation of a polypeptide. Conformational changes generally result from mutations to the gene encoding the polypeptide, but can also occur due to changes in the expression of a co-factor that influences the conformation of the polypeptide. Antibodies that distinguish between two conformations of a polypeptide are known in the art (e.g., there are antibodies known in the art that distinguish the conformation of mutant from wild-type p53).

Changes in post-translational modifications (e.g., phosphorylation, glycosylation, myristoylation, etc.) of a polypeptide can also be useful expression characteristics in diagnosis and/or prognosis of disease.

In some aspects of the invention, sets of cancer-specific markers are used to determine the progression of cancer in a test tissue sample. Perhaps one of the better examples of this application is the diagnosis of small round blue cell tumors in childhood. These tumors show no distinguishing morphological features but require positive identification because of their requirements for specific therapies and clinical outcomes. Immunohistochemistry (IHC) has proven to be one of the most powerful diagnostic tools to help categorize these tumors. In the majority of cases, a carefully selected panel of antibodies can assist in identifying most of the small blue round tumors such as leukemia/lymphoma, Ewing's Sarcoma, rhabdomyosarcoma, and mesenchymal chrondrosarcoma. Although no one specific antibody is diagnostic, each tumor will have a specific pattern of negative and positive antibodies.

Molecular Probes

Antibodies For Detection of Cancer-Specific Markers

Antibodies specific for a large number of known polypeptides are commercially available. Alternatively, or in the case where the expression characteristics of a new growth-related polypeptide is to be analyzed, one of skill in the art can raise their own antibodies. In order to produce antibodies, various host animals are immunized by injection with the growth-related polypeptide or an antigenic fragment thereof. Useful animals include, but are not limited to rabbits, mice, rats, goats, and sheep. Adjuvants may be used to increase the immunological response to the antigen. Examples include, but are not limited to, Freund's adjuvant (complete and incomplete), mineral gels such as aluminum hydroxide, surface active substances such as lysolecithin, pluronic polyols, polyanions, peptides, oil emulsions, keyhole limpet hemocyanin, dinitrophenol, and adjuvants useful in humans, such as BCG (bacille Calmette-Guerin) and Corynebacterium parvum. These approaches will generate polyclonal antibodies.

Monoclonal antibodies specific for a growth-related polypeptide may be prepared using any technique that provides for the production of antibody molecules by continuous cell lines in culture. These include, but are not limited to, the hybridoma technique originally described by Kohler and Milstein, 1975, Nature 256: 495-497, the human B-cell hybridoma technique (Kosbor et al., 1983, Immunology Today 4: 72; Cote et al., 1983, Proc. Natl. Acad. Sci. U.S.A. 80: 2026-2030) and the EBV-hybridoma technique (Cole et al., 1985, Monoclonal Antibodies and Cancer Therapy, Alan R. Liss, Inc., pp. 77-96). In addition, techniques developed for the production of “chimeric antibodies” (Morrison et al., 1984, Proc. Natl. Acad. Sci. U.S.A. 81: 6851-6855; Neuberger et al., 1984, Nature 312: 604-608; Takeda et al., 1985, Nature 314: 452-454) by splicing the genes from a mouse antibody molecule of appropriate antigen specificity together with genes from a human antibody molecule of appropriate biological activity can be used. Alternatively, techniques described for the production of single chain antibodies (U.S. Pat. No. 4,946,778) can be adapted to produce growth-related polypeptide-specific single chain antibodies.

Antibody fragments which contain specific binding sites of a growth-related polypeptide may be generated by known techniques. For example, such fragments include, but are not limited to, F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed (Huse et al., 1989, Science 246: 1275-1281) to allow rapid and easy identification of monoclonal Fab fragments with the desired specificity to a growth-related polypeptide. An advantage of cloned Fab fragment genes is that it is a straightforward process to generate fusion proteins with, for example, green fluorescent protein for labeling.

Antibodies, or fragments of antibodies may be used to quantitatively or qualitatively detect the presence of growth-related polypeptides or conserved variants or peptide fragments thereof. For example, immunofluorescence techniques employing a fluorescently labeled antibody coupled with light microscopic, or fluorimetric detection can be used.

The antibodies or antigen binding fragments thereof may be employed histologically, as in immunofluorescence, immunoelectron microscopy or non-immunoassays, for in situ detection of growth-related polypeptides or conserved variants or peptide fragments thereof.

Detection of Cancer-Specific Markers in Oncology Tissue Microarrays Using Antibodies

In situ detection of a cancer-specific marker can be accomplished by contacting a test tissue and/or an oncology microarray with a labeled antibody that specifically binds the- marker of interest. The antibody or antigen binding fragment thereof is preferably applied by overlaying the labeled antibody onto the microarray and/or test tissue. Through the use of such a procedure, it is possible to determine not only the presence of the cancer specific marker but also its amount and its localization in a test tissue and in the plurality of sublocations within the microarray.

In one aspect, antibodies are detectably labeled by linkage to an enzyme for use in an enzyme immunoassay (EIA) (Voller, 1978, Diagnostic Horizons 2: 1-7, Microbiological Associates Quarterly Publication, Walkersville, Md.); Voller et al., 1978, J. Clin. Pathol. 31: 507-520; Butler, 1981, Meth. Enzymol. 73: 482-523; Maggio, E. (ed.), 1980, Enzyme Immunoassay, CRC Press, Boca Raton, Fla.). The enzyme which is linked to the antibody will react with an appropriate substrate, preferably a chromogenic substrate, in such a manner as to produce a chemical moiety which is detectable, for example, by spectrophotometric, fluorimetric or visual means. Examples of enzymes useful in the methods of the invention include, but are not limited to peroxidase, alkaline phosphatase, and RTU AEC,

Detection of bound antibodies can alternatively be performed using radiolabeled antibodies. Following binding of the antibodies and washing, the samples may be processed for autoradiography to permit the detection of label on particular cells in the samples.

In a preferred aspect, antibodies are labeled with a fluorescent compound. When the fluorescently labeled antibody is exposed to light of the proper wavelength, its presence can be detected due to fluorescence. Many fluorescent labels are known in the art and may be used in the methods of the invention. Preferred fluorescent labels include fluorescein, amino coumarin acetic acid, tetramethylrhodamine isothiocyanate (TRITC), Texas Red, Cy3.0 and Cy5.0. Green fluorescent protein (GFP) is also useful for fluorescent labeling, and can be used to label non-antibody protein probes as well as antibodies or antigen binding fragments thereof by expression as fusion proteins. GFP-encoding vectors designed for the creation of fusion proteins are commercially available.

As mentioned previously, the primary antibody (the one specific for the polypeptide of interest) may alternatively be unlabeled, with detection based upon subsequent reaction of bound primary antibody with a detectably labeled secondary antibody specific for the primary antibody. Another alternative to labeling of the primary or secondary antibody is to label the antibody with one member of a specific binding pair. Following binding of the antibody-binding pair member complex to the sample, the other member of the specific binding pair, with a fluorescent or other label, is added. The interaction of the two partners of the specific binding pair results in binding the detectable label to the site of primary antibody binding, allowing detection. Specific binding pairs useful in the methods of the invention include, for example, biotin:avidin. A related labeling and detection scheme is to label the primary antibody with another antigen, such as digoxigenin. Following binding of the antigen-labeled antibody to the sample, detectably labeled secondary antibody specific for the labeling antigen, for example, anti-digoxigenin antibody, is added and binds to the antigen-labeled antibody, permitting detection.

The staining of tissues for antibody detection is well known in the art, and can be performed with molecular probes including, but not limited to AP-Labeled Affinity Purified Antibodies, FITC-Labeled Secondary Antibodies, Biotin-HRP Conjugate, Avidin-HRP Conjugate, Avidin-Colloidal Gold, Super-Low-Noise Avidin, Colloidal Gold, ABC Immu Detect, Lab Immunodetect, DAB Stain, ACE Stain, NI-DAB Stain, Polyclonal Secondary Antibodies, Biotinylated Affinity Purified Antibodies, HPP-Labeled Affinity Purified Antibodies, and/or Conjugated Antibodies.

Nucleic Acid Probes for Detection of Cancer-Specific Markers

Nucleic acid probes also are useful to correlate the differential expression of genes with abnormal cell proliferation. In one aspect of the invention, the sequences of any of the cancer-specific genes described above are used to generate probes or primers for use in the present invention. Means for detecting specific DNA sequences are well known to those of skill in the art. In one aspect, oligonucleotide probes chosen to be complementary to a selected subsequence within the gene can be used. Alternatively, sequences or subsequences of cells/tissues within a microarray may be amplified by a variety of DNA amplification techniques (e.g., polymerase chain reaction, ligase chain reaction, transcription amplification, etc.) prior to detection using a probe. Amplification of nucleic acid sequences increases sensitivity providing more copies of possible target subsequences. In addition, by using labeled primers in the amplification process, the sequences are as they are amplified.

Methods of labeling nucleic acids are well known to those of skill in the art. Preferred labels are those that are suitable for use in in situ hybridization (e.g., FISH). In one aspect, nucleic acid probes are detectably labeled prior to hybridization with a sample. Alternatively, a detectable label which binds to the hybridization product can be used. Labels for nucleic acid probes include any composition detectable by spectroscopic, photochemical, biochemical, immunochemical, or chemical means and include, but are not limited to radioactive labels (e.g. ³²p, ¹²⁵I, ¹⁴C, ³H, and ³⁵S), fluorescent dyes (e.g. fluorescein, rhodamine, Texas Red, etc.), electron-dense reagents (e.g. gold), enzymes (as commonly used in an ELISA), colorimetric labels (e.g. colloidal gold), magnetic labels (e.g. Dynabeads™), and the like. Examples of labels which are not directly detected include biotin and dioxigenin as well as haptens and proteins for which labeled antisera or monoclonal antibodies are available.

A direct-labeled probe, as used herein, is a probe to which a detectable label is attached. Because the direct label is already attached to the probe, no subsequent steps are required to associate the probe with the detectable label. In contrast, an indirect labeled probe is one which bears a moiety to which a detectable label is subsequently bound, typically after the probe is hybridized with the target nucleic acid.

Labels can be coupled to nucleic acid probes in a variety of means known to those of skill in the art. In some aspects the nucleic acid probes are labeled using nick translation or random primer extension (Rigby et al., 1977, J. Mol. Biol. 113: 237; or Sambrook et al., 1989, Molecular Cloning-A Laboratory Manual, Cold Spring Harbor Laboratory, Cold Spring Harbor, N.Y., the entireties of which are incorporated by reference herein).

Detection of Cancer-Specific Markers Using Nucleic Acid Probes

In situ hybridization (ISH) and Fluorescent In Situ Hybridization (FISH) are techniques that can avail themselves to paraffin-embedded sectioned tissue. Both techniques are genomic based rather than proteomic based as in IHC and involve RNA and DNA probes that will hybridize or specifically bind to their complement base sequence. Markers are attached to the genomic probes that allow the probes to be visualized under a microscope. ISH probes generally have a chromogenic marker and can be observed by traditional light microscopy. FISH probes generally have a fluorescent marker bonded and must be visualized with the use of a fluorescent microscope.

Although IHC markers are most useful in characterizing and identifying tumors, there are some lesions such as endocrine/neuroendocrine tumors in which ISH can add another level of specificity. This is because some tumors may take up proteins nonspecifically or may not be found in levels detectable by IHC. In these instances, genomic probes can be amplified and are more readily detectable. In breast cancer, FISH for the detection of cerbB-2 is commonly used for its strong predictive power.

In one aspect, profile array substrates are used as control tools in ISH/FISH methodologies. Just as in IHC, the option of having 25 individual control specimens simultaneously processed with the test tissue is an extremely valuable tool in probe sensitivity and detection.

For in situ hybridization, sections of paraffin-embedded tissue immobilized on glass substrates are treated as follows, according to one aspect of the invention: Substrates are dewaxed in staining dishes by three changes in xylenes, for 2 minutes each (dewaxing is not necessary for non-embedded single cells). Dewaxed samples are then rehydrated using the following procedure: 100% ethanol, two times, for two minutes, then subsequent 2 minute incubations in 95%, 70%, and 50% ethanol. Samples are denatured by incubation for 20 minutes at room temperature in 0.2 N HCl, followed by heat denaturation for 15 minutes at 70° C. in 2×SSC. Samples are then rinsed in 1×PBS for 2 minutes. In some situations, usually empirically determined, a pronase digestion step may be included here which later allows improved access of the probes to the nucleic acids contained within the tissue sections. In such cases, samples are digested for 15 minutes at 37° C. with predigested, lyophilized pronase at an empirically determined concentration which allows hybridization yet preserves the cellular morphology (0.1 to 10 μg/ml).

Digested samples are incubated for 30 seconds in 2 mg/ml glycine in 1×PBS to stop the digestion. Samples are then post-fixed using freshly prepared 4% paraformaldehyde in 1×PBS, for minutes at room temperature. Fixation is then stopped by a 5 minute incubation in 3×PBS, followed by two 30 second rinses in 1×PBS. Samples are then soaked in 10 mM DTT, 1×PBS, for 10 minutes at 45° C. Samples are then soaked 2 minutes in freshly made 0.1 M triethanolamine, pH 8.0 (triethanolamine buffer). Next, samples are placed in fresh triethanolamine buffer to which acetic anhydride is added to 0.25% final concentration, followed by mixing and 5 minutes' incubation with gentle agitation. After the 5 minutes, more acetic anhydride is added to a final concentration of 0.5%, followed by 5 minutes' further incubation. Samples are blocked 5 minutes in 2×SSC, followed by dehydration through successive soaking in 50%, 70%, 95% (once each), and 100% ethanol (two times) for 2 minutes each at room temperature. Samples are air dried or dried with desiccant before proceeding to the hybridization step. The preceding series of steps may be automated in order to increase throughput.

Probes for in situ hybridization may be DNA or RNA oligonucleotides or, for example, RNA transcribed in vitro. In one aspect, RNA probes labeled with ³⁵S are dissolved in 5 μl of 50 mM dithiothreitol (DTT), and added to 2.5 μl (i.e., an amount approximately equal to one half the mass of labeled probe added) of a non-specific riboprobe competitor (RNA made in the same manner as the labeled specific probe, except from a transcription template with non-specific sequences (for example, vector with no insert) and no labeled ribonucleoside in the reaction). This probe/non-specific competitor mixture is heated at 100° C. for 3 minutes, followed by addition of hybridization buffer (e.g., 50% (v/v) deionized formamide, 0.3 M NaCl, 10 mM Tris (pH 8.0), 1 mM EDTA, 1×Denhardt's solution, 500 mg/ml yeast tRNA, 500 mg/ml poly(A), 50 mM DTT, 10% polyethylene glycol 6000) to 0.3 μg/ml final probe concentration (estimate of amount of probe synthesized is based on calculation of the percent of the label incorporated and the proportion of the labeling base in the probe molecule as a whole).

The probe/hybridization mix is incubated at 45° C. until applied to the microarrays as a thin layer of liquid. Hybridization reactions are then incubated in a moist chamber (closed container containing towels moistened with 50% deionized formamide, 0.3 M NaCl, 10 mM Tris (pH 8.0), 1 mM EDTA) at 45° C. If background proves to be a problem, a 1 to 2 hour pre-hybridization step using only non-specific, unlabeled riboprobe competitor in hybridization buffer can be added prior to the step in which labeled probe is applied.

Hybridization is carried out for 30 minutes to 4 hours, followed by washing to remove the unbound probe. The microarrays are washed in an excess (100 ml each wash) of the following buffers: 50% formamide, 2×SSC, 20 mM β-mercaptoethanol, two times, for 15 minutes at 55° C.; 50% formamide, 2×SSC, 20 mM β-mercaptoethanol, 0.5% Triton X-100, two times, for 15 minutes at 55° C.; and 2×SSC, 20 mM β-mercaptoethanol, two times, for 2 minutes at 50° C.

The samples are then subjected to an RNAse digestion for 15 minutes at room temperature using a solution containing 40 mg/ml RNase A, 2 mg/ml RNase TI, 10 mM Tris (pH 7.5), 5 mM EDTA and 0.3 M NaCl. After RNase digestion, slides are soaked two times for 30 minutes each in 2×SSC, 20 mM β-mercaptoethanol at 50° C., followed by two washes in 50% formamide, 2×SSC, 20 mM β-mercaptoethanol at 50° C. and two washes of 5 minutes each in 2×SSC at room temperature. Hybridized, washed substrates comprising microarrays are dehydrated through successive two minute incubations in the following: 50% ethanol, 0.3 M ammonium acetate; 70% ethanol, 0.3 M ammonium acetate; 95% ethanol, 0.3 M ammonium acetate; 100% ethanol. Substrates are air dried overnight, followed by coating with emulsion for autoradiography according to standard methods.

Sections prepared, for example, from frozen tissues, may be hybridized by a similar method except that the dewaxing and paraformaldehyde fixation steps are omitted. For details, see Ausubel et al., 1992, Short Protocols in Molecular Biology, John Wiley and Sons, Inc., pp. 14-15 to 14-16.

As an alternative to the so-called “conventional” in situ hybridization methods described above and in the references therein, the method of in situ PCR can be used to examine the presence of nucleic acids encoding a growth-related polypeptide in cells, tissues, or other sample preparations in which the levels of such nucleic acids are low. A detailed description of the technique is presented in Ausubel, et al., 1992, supra, pp. 14-37 to 14-49, the contents of which are hereby incorporated by reference.

In a further aspect of the invention, ISH or FISH probes or other nucleic acid molecular probes (e.g., DAPI, acridine orange) are used to evaluate the absolute amounts of nucleic acids in cells within a tissue (e.g., to determine the copy number of nucleic acids on the tissue); changes in copy number of nucleic acids are often associated with the development of pathology.

In still a further aspect of the invention, information obtained from a single sublocation can be combined by combining the detection of both proteins and nucleic acids. For example, in one aspect of the invention, after performing immunohistochemistry on cells/tissue at a sublocation, a portion of the sample is obtained to isolate nucleic acids which are further analyzed by amplification methods such as PCR. Detection of nucleic acids isolated from an embedded cell or tissue sample is known in the art and is described in, for example, U.S. Pat. No. 6,013,461, U.S. Pat. No. 6,110,902, and U.S. Pat. No. 6,114, 110, the entireties of which are incorporated by reference herein.

Adaptation of these procedures to the specific needs of varying sample types is within the ability of those with ordinary skill in the art and those of skill in the art may select and employ appropriate and routine methods of detection (Ausubel et al., 1992, supra, pp. 14-18 to 14-19, describes autoradiographic detection) and counterstaining of the tissue sections (e.g., with hematoxylin/eosin, among others, described in Ausubel et al., 1992, supra, pp. 14-19 to 14-22) to make hybridization signal and cell and tissue morphology readily apparent using visual inspection, microscopy, or either visual inspection or microscopy enhanced through the used of optical systems placed in communication with the microarrays according to the invention.

Scoring Method for Classifying Biological Characteristics

In one aspect, a panel or collection of cell and/or tissues samples is obtained representing a plurality of different stages of cancer which is used to generate the sublocations of an oncology microarray. In order to establish a panel which is useful for predicting the prognosis of a given cell or tissue sample, a scoring method is established which relates the expression of a first biological characteristic (e.g., level of expression cancer-specific marker, as reflected by antibody staining) to a second biological characteristic (e.g., localization of the cancer-specific marker). Thus, in one aspect, the biological marker is nuclear staining for the polypeptide, and the tissue collection is classified according to the percentage of cells expressing the polypeptide and how intensely those cells express the polypeptide. Cancer cells are placed into groups based on 1) a range of percentages of cells expressing the marker polypeptide, for example, 5 groups of <20%, 20% to <40%, 40% to <60%, 60% to <80%, and 80% to 100%, and 2) a range of degrees of staining intensity, for example, 4 groups ranging from light staining, light to medium staining, medium to dark staining and dark staining.

These quantities are used to place the expression characteristic for a given test sample into one of a number of categories that considers both elements of the characteristic being classified. The number of categories in this case is determined as the product of the number ranges of percentages and the number of ranges of staining intensity (in the present example, there would be 20 categories; a single further category can be added that includes cancer cells with no nuclear staining for the polypeptide). The categories are illustrated below in Table 1. In reference to the table, for example, a sample with 35% of cells staining light to medium would be scored 2/2. One should also note that within a given tissue sample there are most frequently more than one cell type. The scoring of cells in the tissue samples can be done individually in those cases in which the tumor retains morphologically distinct cell types. Thus, for a given tissue sample, one may have separate expression characteristic scores for, e.g., epithelial cells, glandular cells and inflammatory cells; or other indicia of morphology that reflect any of the grading systems for abnormal cell growth described above (e.g., TNM, Duke's stage, Gleason stage, BRE stage, and the like). By correlating the matrix data (e.g., as in the Table below) with the grade of cancer, a user of the microarray can stage a test tissue by identifying the two biological characteristics expressed in the tissue.

TABLE 1
Percent (%) of Cells Staining

| Degree of | | | | | |
|---|---|---|---|---|---|
| Staining | <20% | 20%-<40% | 40%-60% | 60%-<80% | 80%-100% |
| Light | 1/1 | 1/2 | 1/3 | 1/4 | 1/5 |
| Light/ | 2/1 | 2/2 | 2/3 | 2/4 | 2/5 |
| Medium | | | | | |
| Medium/ | 3/1 | 3/2 | 3/3 | 3/4 | 3/5 |
| Dark | | | | | |
| Dark | 4/1 | 4/2 | 4/3 | 4/4 | 4/5 |

Thus, when the score assigned to a patient's tissue sample for a given biological characteristic (e.g., a cancer-specific marker) substantially matches the score of a test sample for the same biological characteristic (i.e., is not statistically different based on routine statistical tests to within 95% confidence levels), the prognosis of the patient's disease is correlated to that of the patient from whom the standard sample was obtained. The accuracy of prognosis value increases as more markers are considered. Thus, the ability to screen serial sections of the cell/tissue microarray with multiple probes, and to correlate the expression characteristics of biomolecules reactive with those probes on a single microarray with the same probes on another microarray, facilitates the generation of an expression profile representing multiple biological characteristics. These profiles are useful in diagnosis, prognosis, guidance of treatment and prediction of a patient's relapse.

Information relating to a diagnostic matrix established for a given type of cancer and a given microarray is stored in a database, along with all other information available relating to the patient from which a particular tissue sample came. However, in addition to the information regarding each tissue sample on the panel, the database can contain information on other tissue samples not included on the particular array (or arrays) examined by a given clinician. These data provide depth to the database beyond the samples on a given array, and enhances the statistical reliability of decisions based upon a given array. For example, a collection of 250,000 or more samples of breast cancer tissue may be available. A given tissue array will not necessarily have samples of all of them, but will more likely have a subset of those tissue samples. Therefore, there can be multiple arrays, each comprising a different subset of the total collection of samples. As each subset array is analyzed for different markers, the data are reported back to the database. When a clinician reports data back to the database for a given marker, they can be informed of whether other clinicians have examined the same marker in other samples on other subset arrays.

The information for those subset arrays examined for the same marker can then be provided to the clinician for use in diagnosis or prognosis of their patient's condition. The result of this is that examination of an array of, for example, 500 tissue samples can effectively yield information on many more tissue samples in other subset arrays. The predictive value of a standard panel and the database associated with it increases as data is reported back to the database for individual markers.

Selecting Promising Gene Targets and Validating Diagnostic Molecules Using Oncology Tissue Microarrays

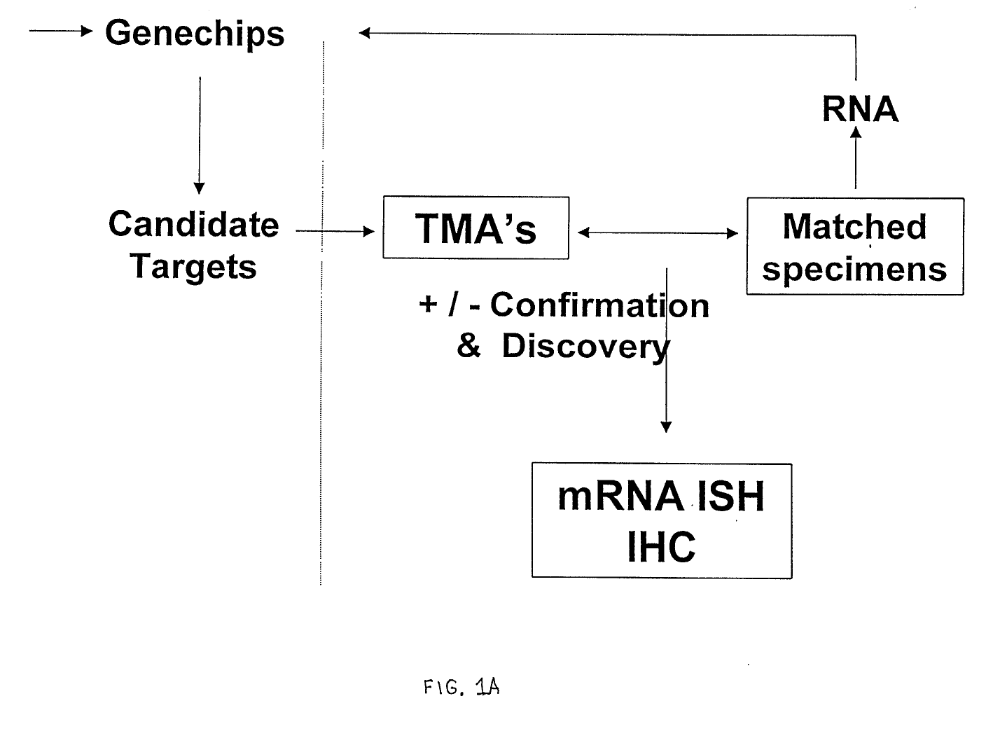

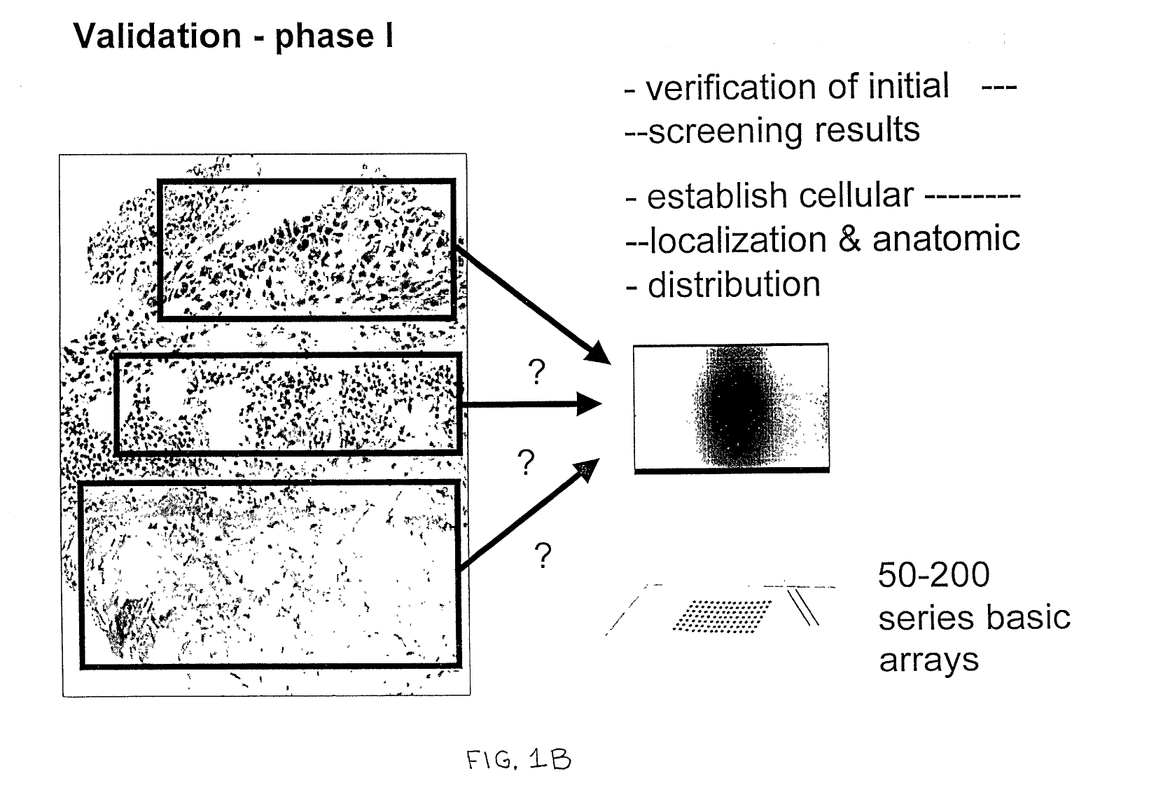

In one aspect, test probes specifically reacting with a gene or gene product are used to identify candidate drug targets (see FIGS. 1A-B). Test probes can include antibodies, nucleic acids, aptamers, enzymes, substrates, and the like, and are obtained by screening one or more of a nucleic acid array (e.g., oligonucleotide arrays, cDNA arrays, Expressed Sequence Tag Arrays), a peptide, polypeptide, protein array, or other small molecule array, with a patient sample to identify a biomolecule or set of biomolecules whose expression is diagnostic of a trait (e.g., by determining which molecules on the array are substantially always present in a disease sample and substantially always absent in a healthy sample, or substantially always absent in a disease sample and substantially always present in a healthy sample, or substantially always present in a certain form/amount in a disease sample and substantially always present in a certain other form/amount in a healthy sample). As used herein “substantially always” refers to a statistically significant difference between samples from healthy patients and patients having a disease.

Test probes identifying diagnostic biomolecules are then contacted with an oncology microarray according to the invention, to identify the presence and/or form of diagnostic biomolecules in a microarray comprising different types of healthy or diseased tissues. In this way, the correlation between the expression of the diagnostic biomolecule(s) and the disease state is validated.

In another aspect of the invention, the role of the diagnostic molecule(s) are evaluated by comparing the expression of the molecule(s) in different sublocations on the microarray(s) with information in a database relating to the type of tissue, its developmental stage, or to other traits of the individual(s) from which the tissue is obtained (e.g., patient information).

In a further aspect of the invention, the expression of diagnostic molecules is examined in a microarray comprising samples from a drug-treated patient and samples from an untreated diseased patient and/or from a healthy patient, and the efficacy of the drug is monitored by determining whether the expression profile of the diagnostic(s) molecule returns to that of a healthy patient. In one aspect of the invention, a test tissue which is the target of a disease is obtained from a patient treated with a drug and a microarray is provided which comprises tissue which is the target of disease from a healthy patient and from a patient with the disease. The expression of diagnostic molecule(s) in the test tissue is compared with the expression pattern of these molecules in the target tissues in the microarray. A drug is identified as useful for further testing when the expression pattern in the test tissue is substantially the same as the expression pattern within the healthy tissue (to within 95% confidence levels). Preferably, one or more tissues which are not the target of the disease also are arrayed and the expression of biomolecules in corresponding test tissues from drug-treated patients, non-drug treated diseased patients, and non-drug treated healthy patients, to evaluate whether the drug has non-specific effects on biomolecules in tissues other than the target of disease.

Kits

Kits are contemplated for use in the methods according to the invention including any of the oncology tissue microarrays described above, profile array substrates comprising the microarrays, and a means for providing access to information on each cell/tissue sample at each sublocation, the information including, but not limited to, full pathology and clinical data, including medications and treatment history of the individual from whom the tissue was obtained. In one aspect of the invention, a kit includes any or all of breast cancer tissue progression microarray comprising at least about 20 sublocations comprising different breast tumor types, and including about 5 normal sublocations; a colon cancer progression microarray comprising at least about 20 sublocations comprising different colon tumor types, and including about 5 normal sublocations; a prostate cancer progression microarray comprising at least 20 sublocations comprising different prostate tumor types, and including about 5 normal sublocation; a normal tissue microarray comprising different types of tissue; a tumor microarray comprising different tumors obtained from different tissue types, including at least one normal sublocation; and a non-human animal (e.g., rat or mouse) microarray comprising sublocations representing different tissue types, at least one tissue type comprising abnormally proliferating cells. In one aspect, cells/tissues from non-human animals genetically engineered to comprise one or more cells having less than two or more than two copies of a gene involved in cell proliferation or cell death, or to express modified forms of such genes.

In one aspect, low density microarrays are provided which comprise over about 45-60 sublocations per slide.

In a further aspect, a kit is provided comprising high density tissue microarrays which represent population surveys of normal and clinical conditions for the evaluation of gene expression patterns. In one aspect, the microarrays comprise over 200 tissue samples. In one aspect, a kit is provided comprising a cancer screening array comprising over 200 samples of normal and cancer tissue, and a standard control microarray comprising samples from the following organs: liver, lymph node, kidney, hyroid and prostate. In a further aspect, a kit is provided comprising a plurality of microarrays including one or more of a normal tissue microarray, a breast cancer progression microarray, prostate cancer progression microarray, a colorectal cancer progression microarray, a lung cancer progression microarray, and/or a cancer screening microarray described.

In still a further aspect of the invention, a kit is provided comprising microarrays comprising one or more samples pre-reacted with a labeled molecular probe or stain. For example, samples pre-reacted with labeled molecular probes which specifically react with a molecule selected from the group consisting of actin, CEA, chromogranin, desmin, EMA, GFAP, HMB, MSE, PLAP, PSA, PSAP and vimentin can be provided.

In other aspects, the following antigen-specific probe/tissue combinations are provided: Actin, normal colon; Actin, uterine smooth muscle; Carcinoembryonic antigen (CEA), colon adenocarcinoma; CD3, T-cell, tonsil; CD15/LeuM1, lymphoma; CD20/L26, B-cell, tonsil; CD30/BerH2, lymphoma; CD34, hematopoietic progenitor cells, tonsil; CD34, hematopoietic progenitor cells, normal skin; CD45/LCA, T-cell, tonsil; CD68/KPI, macrophage, tonsil; Chromogranin A, pancreas; Chromogranin A, pancreas slides; Cytokeratin, pan-keratin, normal prostate; Cytokeratin, pan-keratin, normal skin; Cytokeratin 7, breast ductal carcinoma; Cytokeratin 20, colon adenocarcinoma; Cytokeratin 20, bladder carcinoma; and Cytokeratin, high molecular weight, skin; Cytokeratin, high molecular weight, prostate; Desmin, leiomyoma; Desmin, normal colon; Epithelial membrane antigen (EMA), meningioma; Epithelial membrane antigen (EMA), breast; Glial fibrillary acidic protein (GFAP), brain; HMB45, melanosome, melanoma; HMB45, melanosome, melanoma, Clark score 1-5 w/nevus; Kappa light chains, tonsil; Lambda light chains, tonsil; Neuron specific enolase (NSE), pancreas; Placental alkaline phosphotase (PLAP), seminoma; Prostate specific antigen (PSA), prostate; 1132-5 Prostatic acid phosphotase (PSAP), prostate; S 100, skin; S100, melanoma; Vimentin, tonsil; Vimentin, normal colon;Von Willebrand factor (Factor VIII), tonsil; Estrogen receptor, breast carcinoma; Progesterone receptor, breast carcinoma.

In a further aspect of the invention, the kit can comprise genomic DNA from one or more of bladder, brain, breast, cervix, colon, esophagus, heart, small intestine kidney, liver, lung, skeletal muscle, pancreas, prostate, rectum, skin, spleen, stomach, testis, and the like.

Additional reagents and kit components include, but are not limited to, antibodies, labels, DNA or RNA probes, and the like.

### EXAMPLES

The invention will now be further illustrated with reference to the following examples. It will be appreciated that what follows is by way of example only and that modifications may be made while still falling within the scope of the invention.

### Example 1

### Normal Tissue Microarray (NO50 and NO200)

This tissue microarray is designed for identification of normal tissue types where expression of a particular gene/gene product, or other genetic alteration or biomarkers occurs.

Composition

The NO50 Normal tissue microarray contains 4 samples each of 20 different tissue types all on a single microarray. For each tissue type, the samples are derived from multiple different individuals (2-4 individuals) and include: cerebrum, grey substance, cerebellum, heart, lung, thyroid gland, adrenal gland, pancreas, liver, tonsil, spleen, lymph node, endometrium, secretion, ovary-stroma, myometrium, placenta-third trimenon, kidney-cortex, prostate, seminal vesicle, and skeletal muscle. Patient information and information relating to other biological characteristics of each tissue in the microarray are stored in a specimen-linked database. Information includes site of biopsy, tissue represented, histological diagnosis, underlying disease, age at time of diagnosis, source of tissue (e.g., from biopsy or from autopsy), in case of autopsies, the time span between death and autopsy is also provided (see Table 10, for example). The microarray is also provided with an array locator to address the sublocations on the array.

Normal Tissue Microarray (NO200)

The N0200 set comprises a plurality of microarrays including: 2 NO200 Normal tissue array sections, 3 TE30 Test slides (described in Example 2), 1 NO200 data report, 1 TE30 data report and the NO200 database, of the associated pathology and clinical data (see Table 11).

Composition

The NO200 Normal tissue microarray contains 10 samples each of 40 different tissue types. For each tissue type the samples are derived from multiple different individuals (2-6 individuals) and include: cerebrum-grey substance, -cerebrum, white substance, cerebellum, heart, bronchus, lung, thyroid gland, adrenal gland, skin, pancreas, submandibular gland, stomach-corpus, stomach-antrum, duodenum, ileum, appendix, colon, liver, gall bladder, tonsil, spleen, lymph node, ovary-stroma, ovary-corpus luteum, fallopian tube, endometrium-proliferation, endometrium secretion, endocervix, ectocervix, myometrium, placenta (last trimenom), kidney cortex, kidney-papilla, kidney-pelvis, prostate, seminal vesicle, testis, epidydimis, skeletal muscle, and smooth muscle.

Exemplary data relating to the biological characteristics of tissue samples in the microarray which are stored in the database is provided in Tables 2-22, in accompanying, Appendix A, the entirety of which is incorporated by reference herein. The coordinates of the sublocations on the microarray are defined using an array locator.

### Example 2

### Oncology Test Array (TE30)

This microarray been designed to find optimal conditions for molecular analysis to be performed on larger microarrays.

Composition

The TE30 test array contains a total of 30 tissue samples of the following types: colon cancer (n=5);-breast cancer (n=5); lung cancer (n=5); prostate cancer (n=5); normal tissues from the following organs: liver, skeletal muscle, lymph node, kidney cortex, thyroid, prostate, spleen.

Exemplary data relating to the biological characteristics of tissue samples in the microarray which are stored in the database is provided in Table 3, in accompanying Appendix A. The coordinates of the sublocations on the microarray are defined using an array locator.

### Example 3

### Head and Neck Cancer Progression Array

HN200

This oncology tissue microarray is designed do find associations between expression of a particular gene/gene product, other genetic alterations or biomarkers and different stages of head and neck cancer progression.

Composition

The HN200 head and neck cancer array contains a total of 200 tissue samples (each at two different sublocations on the array): normal oral mucosa from patients with no history of head and neck cancer (10 sublocation), paired tissues: normal and cancerous oral mucosa from same patients (20 sublocations), oral mucosa with mild to moderate dysplasia (20 sublocations) oral mucosa with severe dysplasia/carcinoma in situ (10, sublocations), nodal negative head and neck cancer (80 sublocations), nodal positive head and neck cancer (80 sublocations), paired metastases: lymph node metastases from tumors included in this microarray (40 sublocations), standard control section of the array contains normal tissues from brain, heart, liver, spleen, muscle, lymph node, testis, kidney, thyroid, adrenal gland, lung and prostate (24 sublocations).

Head and Neck Cancer Screening Array (NH50)

This oncology tissue microarray has been designed to identify whether expression of a particular gene/gene product, other genetic alterations occur in head and neck cancer.

Composition

The HN50 head and neck cancer array contains a total of 50-80 tissue samples (with duplicate sublocations): head and neck cancers of different locations/stages (30-60 sublocations), standard control section of the array contains normal tissues from oral mucosa, liver, spleen, lymph node, kidney, thyroid, and prostate (20 sublocations), and can also include, normal oral mucosa: tonsils, and cancers of: lip, tongue, tonsil, oral, pharynx.

### Example 3

### Prostate Cancer Progression Array Set—PR200

This Prostate Cancer Progression Array Set has been designed to find associations between molecular events and different stages of prostate cancer progression and comprises a set of microarrays.

Composition

The PR200 set contains: 2 PR200 Prostate cancer progression array sections, 3 TE30 Test slides, 1 PR200 data report, 1 TE30 data report, the PR200 database, of the associated pathology and clinical data.

The PR200 Prostate Cancer Progression Array contains a total of 200 tissue samples of the following types (double spotted): benign prostatic hyperplasia, prostatic intraepithalial neoplasia (PIN; high grade), prostate cancer (Gleason score 1-2), prostate cancer (Gleason score 3), prostate cancer (Gleason score 4), prostate cancer (Gleason score 5), prostate cancer metastases, standard control section of the array contains normal tissues from the following organs: brain, heart, liver, spleen, muscle, lymph node, testis, kidney, thyroid, adrenal gland, lung and prostate.

### Example 4

### Prostate Cancer Survey Array—PR50

The Prostate Cancer Survey Array has been designed as a first line screening tool to see whether expression of a particular gene, gene product, or other genetic alteration or biomarker occurs in prostate cancer.

Composition

The PR50 microarray set contains: 4 PR50 Prostate cancer survey array sections, 1 H&E Stained prostate cancer survey array section, 1 PR50 data report, PR50 database including associated pathology and clinical data.

The PR50 Prostate Cancer Survey Array contains a total of 60-80 tissue samples of the following types: prostate cancer (45-60 sublocations); standard control section contains samples from the following organs: prostate, liver, lymph node, kidney, thyroid and seminal vesicles. Data relating to sublocations on the array is included in Table 4 in Appendix A.

Table 18 in Appendix A shows results of screening Prostate and Normal Tissue arrays using a molecular probe (e.g., an antibody) reactive with a-testosterone. Staining in the epithelium and/or stroma is correlated with coordinates on the microarray as well as patient sex, age, organ, tumor type, stage of cancer, and source (e.g., surgery). Tables 20-22 provide data relating to the reactivity of the probe in cancerous but non-prostate tissues as well as prostate and normal tissues using frozen tissue microarrays. The information from these Tables is stored in the specimen-linked database.

### Example 5

### Colorectal Cancer Progression Array (CR200)

The Colorectal Cancer Progression Array set has been specifically designed to find associations between expression of a particular gene, gene product, other genetic alterations or biomarkers and the different stages of colorectal cancer progression.

Composition

The CR200 set contains: 2 CR200 Colorectal cancer progression array sections, 3 TE30 Test slides, 1 CR200 data report, 1 TE30 data report, the CR200 database, of the associated pathology and clinical data.

The CR200 Colorectal Cancer Progression Array contains a total of 200 tissue samples (double spotted) of the following types: normal colon mucosa from patients having no history of colorectal cancer, paired tissues: normal and cancerous colon mucosa from same patients, adenoma with mild dysplasia, adenoma with moderate dysplasia, adenoma with severe dysplasia, nodal negative colorectal cancer, nodal positive colorectal cancer, paired metastases: lymph node metastases from tumors included in this TMA and a standard control section of the array contains normal tissues from the following organs: brain, heart, liver, spleen, muscle, lymph node, testis, kidney, thyroid, adrenal gland, lung, and prostate.

A smaller microarray (CR50) providing a total of 50 tissue samples is described in Tables 5 and 6 and can be used in screening or validation of target biomolecules.

### Example 6

### Cancer Screening Array—CS200

The Cancer Screening Array according to one aspect of the invention has been specifically designed to survey multiple cancer types for the identification of tumor types that express a particular gene, gene product, genetic alteration or other biomarker.

Composition

The CS200 set contains: 2 CS200 Cancer screening array sections, 3 TE30 Test slides, 1 CS200 data report, 1 TE30 data report, and access to the CS200 database, of the associated pathology and clinical data (see Table 7).

The CS200 Cancer Screening Array contains a total of 200 tissue samples from a number of different tumor types: colorectal cancer, prostate cancer, lung cancer, breast cancer, kidney cancer, urinary bladder cancer, ovarian cancer, brain tumors, malignant melanoma, head and neck cancer and a standard control section of the array which contains normal tissues from the following organs: brain, heart, liver, spleen, muscle, lymph node, testis, kidney, thyroid, adrenal gland, and lung.

Results from screening such an array with a molecular probe reactive with immunophilin (α-FKBP51) which is suspected of modulating steroid receptor responses are shown in Table 15 of Appendix A. Reactivity of the microarray with the probe is correlated with age, sex, tumor type, grade, lymph node status, DM status, source (e.g., surgery or biopsy) and resection margins and this information is stored in the specimen-linked database.

### Example 7

### Lung Cancer Progression Array—LU200

The Lung Cancer Progression Array has been designed to find associations between molecular events and different histologic subtypes and stages of lung cancer.

Composition

The LU200 set contains: 2 LU200 Lung cancer progression array sections, 3 TE30 Test slides, 1 LU200 data report, 1 TE30 data report, the LU200 database, of the associated pathology and clinical data.

The LU200 Lung Cancer Progression Array contains a total of 200 tissue samples of the following types (double spotted): normal lung parenchyma, normal bronchi (epithelium), adenocarcinoma (different subtypes), squamous cell carcinoma, adenocarcinoma, undifferentiated large cell carcinoma, small cell carcinoma, lymph node metastases from tumors included in this microarray (paired metastases), standard control section of the array contains normal tissues from the following organs: brain, heart, liver, spleen, muscle, lymph node, testis, kidney, thyroid, adrenal gland, and prostate.

### Example 8

### Cervical Cancer Progression Microarray

The Cervical Cancer Array has been designed to find associations between molecular events and different histologic subtypes and stages of cervical cancer. The tissues represented in this array, their locations, and associated patient information is provided in Table 8, in Appendix A.

### Example 9

### Breast Cancer Progression Microarray

The Breast Cancer Array has been designed to find associations between molecular events and different histologic subtypes and stages of breast cancer. The tissues represented in this array, their locations, and associated patient information is provided in Table 9, in Appendix A.

### Example 10

### Uterine and Ovarian Carcinoma Test

CA125 (receptor-binding cancer antigen expressed in SiSo cells) is a novel tumor associated antigen expressed in human uterine and ovarian carcinomas. The predicted amino acid sequence of CA125 (213a.a.) possesses an N-terminal transmembrane region and a coiled-coil structure in the C-terminal portion, indicating that CA125 is a type II membrane protein able to form oligomers through the coiled-coil structure. CA125 revealed different expression pattern from the known tumor associated antigens such as YH206, GA733, CA125, CEA and sialyl Le molecules in human tumor cell lines. Recent studies indicate that CA125 acts as a ligand for a putative receptor present on various human cells including T, B, and NK cells. CA125 inhibits the in vitro growth of receptor-expressing cells and induced apoptosis. It has been suggested that tumor cells might evade immune surveillance by expression of CA125.

Anti-CA125 antibody can be obtained purified from mouse ascites fluid using protein-L Sepharose. Monoclonal antibodies can be obtained from hybridomas established by fusion of mouse myeloma cell x63. Ag8.653 with Balb/c splenocyte immunized with human uterine cervical adenocarcinoma cells. The antibody is preferably tested by flow cytometry and/orimmunohistochemical staining. The antibody may be used for immunohistochemical analysis of ovarian, cervical, or endometrial adenocarcinoma.

Immunohistochemical staining of paraffin sections can be done as follows:

1. Deparaffinize section, hydrate to water (Xylene-3 times, Ethanol-3 times, PBS-3 times)

2. Wash in PBS for 5 minutes before starting the stain.

3. Remove slides from PBS and cover each with 100 to 200 microliters of 3% H202 for 10 minutes to block endogenous Peroxidase activity. Wash in PBS twice for 5 minutes each.

4. Remove slides from PBS, wipe gently around each section and cover tissue with 100 to 200 microliters of protein blocking reagent for 5 minutes.

5. Tip off the blocking reagent, wipe gently around each section and cover tissue with 100 to 200 microliters of primary antibody CA125 atan about 1:500 dilution

6. Incubate for 1 hour at room temperature.

7. Wash slide with a stream of PBS from a wash bottle. Wash in PBS 3 times for 5 minutes each.

8. Wipe gently around each section and cover tissue with 100 to 200 microliters of polyvalent biotinylated antibody

9. Incubate for 30 min at room temperature.

10. Wash as in step 7.

11. Wipe gently around each section and cover tissue with 100 to 200 microliters of strepavidin conjugated HRP.

12. Incubate for 30 minutes at room temperature.

13. Wash as in step 7.

14. Visualize with DAB substrate/chromogen (20 mg of DAB in 400 ml of PBS containing 40 microliters 3% H2O2) for 15 min. Wash in distilled H2O

15. Counterstain in Hematoxylin for 1 min

16. Wash in PBS.

17. Dehydrate and clear using ethanol and xylene.

18. Mount coverglass.

Results of such an evaluation are shown in Table 16 which correlates reactivity of an anti-CA125 molecular probe with information such as patient sex, age, organ, tumor type, grade, lymph node status, DM status, source (e.g., surgery or biopsy), and resection margins, and coordinates on a tissue microarray. Table 17 additionally correlates reactivity with diagnosis. Table 19 provides data from ovarian, endometrial and cervical carcinomas. The information in the Tables is obtained from the specimen-linked database.

### Example 11

### Diagnosing Lymph Node Positive Biopsies

Lymph node biopsies with known follow-up outcomes as non-metastatic (NM) or metastatic (M) are diagnosed using both morphological methods and IHC using an anti-bcl-2 antibody as a molecular probe after arraying the same on tissue microarrays. In one aspect, negative lymph node biopsies (determined to be negative based on morphological criteria) are examined to determine false negative rates using both reactivity to a bcl-2 specific molecular probe and morphological criteria. Data relating to reactivity is stored in a specimen-linked database in which the identity/location of biopsy tissue on the array is correlated to clinical data regarding cancer outcome and/or progression. The suitability of bcl-2 as a diagnostic probe is determined using the tissue information system according to the invention.

### Example 12

### Molecular Profiling of Breast Cancer Tissue

Table 12 and 13 of Appendix A show results of molecular profiling assays of breast tissue from 356 different patients. Table 12 provides a summary of pertinent clinical information stored in the specimen-linked database. Table 13 shows the results of reacting tissue microarrays comprising breast tissue with anti-Her-2/Neu probes and anti-Estrogen Receptor (ER probes). Samples 1-180 are non-metastatic and show good prognosis while samples 181-360 are metastatic and show poor prognosis. Table 14 shows a dataset from different microarrays correlating position on the array (“localization”) with biological characteristics of the tissue sample including histologic type and subtype, BRE grade, polymorphy in the sample, mitoses, diameter, lymph node positive or negative status, and patient information, including age and survival data.

Variations, modifications, and other implementations of what is described herein will occur to those of ordinary skill in the art without departing from the spirit and scope of the invention.

What is claimed is:

1. An oncology microarray comprising a plurality of samples, each sample stably associated with a distinct, known sublocation on a substrate, at least one sample comprising abnormally proliferating cells, the substrate further comprising an identifier providing access to a database comprising information relating to at least one patient from whom at least one sample was obtained.

2. The microarray according to claim 1, wherein said identifier, when entered into the interface of a user computer connectable to the network, causes a screen to be displayed displaying one or more links to portions of the database comprising information relating to said at least one patient.

3. An oncology microarray comprising a plurality of samples, each sample stably associated with a distinct, known sublocation on a substrate, at least one sample comprising abnormally proliferating cells, and at least one sample comprising frozen cells or tissue.

4. An oncology microarray comprising a plurality of samples, each sample stably associated with a distinct, known sublocation on a substrate, at least one sample comprising abnormally proliferating cells, and at least one other sample comprising cells from a bodily fluid from the same patient providing the sample of abnormally proliferating cells.

5. The microarray according to any of claims 1, 3, or 4, wherein at least one sample comprises normally proliferating cells.

6. The microarray according to any of claims 1, 3, or 4, comprising at least one sample selected from the group consisting of cancerous breast tissue, cancerous prostate tissue, cancerous colon tissue, cancerous cervical tissue, skin cancer, and cancerous lung tissue.

7. The microarray according to any of claims 1, 3, or 4, wherein at least about 10% of the samples of the microarray are from different tissue types.

8. The microarray according to any of claims 1, 3, or 4 comprising samples from at least about five different tumor types.

9. The microarray according to any one of claims 1, 3 or 4, wherein at least one sample is greater than about 0.6 mm in diameter.

10. The microarray according to claim 3, further comprising at least one sample comprising paraffin- or plastic-embedded cells or tissue.

11. The microarray according to any of claims 1, 3 or 4, comprising a plurality of samples representing different grades or stages of a single type of cancer.

12. The microarray according to claim 11, wherein said cancer is selected from the group consisting of breast cancer, skin cancer, head and neck cancer, colon cancer, cervical cancer, ovarian cancer, prostate cancer, and lung cancer.

13. The microarray according to claim 1, 3, or 4, comprising a least one sample which comprises substantially homogeneous cells.

14. The microarray according to claim 13, wherein the at least one sample which comprises substantially homogeneous cells comprises cells which express a cancer-specific marker.

15. The microarray according to any of claims 1, 3, or 4, wherein at least one sample is from a patient treated with a drug.

16. The microarray according to any of claims 1, 3, or 4, wherein at least one sample is from a site of secondary metastasis of a cancer.

17. A kit comprising:
(a) an oncology microarray, said oncology microarray comprising a plurality of samples, each sample stably associated with a distinct, known sublocation on a substrate, at least one sample comprising abnormally proliferating cells; and
(b) a normal tissue microarray, comprising at least two samples each of at least about two different tissue types.

18. A kit according to claim 17, wherein said normal tissue microarray comprises at least about five different tissue types.

19. The kit according to claim 18, wherein said five different tissue types are selected from the group consisting of cerebrum, cerebellum, medulla, cardiac tissue, lung tissue, thyroid gland, adrenal gland, submandibular gland, pancreas, liver, tonsil, spleen, lymph node, endometrium, ovary stroma, myometrium, fallopian tube, endocervix, ectocervix, placenta, kidney, prostate, seminal vesicle, stomach tissue, duodenum, ileum, appendix, colon, gall bladder, skeletal muscle, and smooth muscle.

20. The kit according to claim 18, wherein said at least about five different tissue types are from a single patient.

21. The kit according to claim 18, wherein said normal tissue microarray comprises at least about ten different tissue types.

22. The kit according to claim 21, wherein said normal tissue microarray comprises at least about twenty different tissue types.

23. The kit according to claim 22 wherein said oncology array or said normal array further comprises an identifier providing access to a database comprising information relating to at least one patient from whom at least one sample was obtained.

24. The kit according to claim 23, wherein said identifier, when entered into the interface of a user computer connectable to the network, causes a screen to be displayed displaying one or more links to portions of the database comprising information relating to said at least one patient.

25. The kit according to claim 24, wherein said oncology microarray further comprises at least one sample comprising frozen cells or tissue.

26. The kit according to claim 24, wherein said oncology microarray further comprises at least one other sample comprising cells from a bodily fluid from the same patient providing the sample of abnormally proliferating cells.

27. The kit according to claim 24, wherein said oncology microarray comprises cells from abnormally proliferating head and neck tissue.

28. The kit according to claim 27, wherein said oncology microarray comprises one or more of normal oral mucosa, cancerous oral mucosa with mild to moderate dysplasia, oral mucosa with severe dysplasia or carcinoma in situ, nodal negative head and neck cancer, nodal positive head and neck cancer, and lymph node metastases.

29. The kit according to claim 27, wherein said oncology microarray comprises one or more samples comprising cancers of the lip, tongue, tonsil, oral mucosa, and pharynx.

30. The kit according to claim 24, wherein said oncology microarray comprises one or more samples from tissues comprising benign prostatic hyperplasia, prostatic intraepithelial neoplasia, prostate cancer (Gleason score 1-2), prostate cancer (Gleason score 4), prostate cancer (Gleason score 5), and prostate cancer metastases.

31. The kit according to claim 24, wherein said oncology microarray comprises one or more samples from normal colon tissue, cancerous colon mucosa, adenocarcinoma with mild dysplasia, adenoma with severe dysplasia, nodal negative colorectal cancer, nodal positive colorectal cancer, and colon cancer metastases.

32. The kit according to claim 24, comprising one or more samples from normal lung parenchyma, normal lung bronchi, adenocarcinoma, squamous cell carcinoma, undifferentiated large cell carcinoma, and metastases from cancerous lung tissue.

33. The kit according to claim 24, wherein said oncology microarray comprises one or more normal breast tissue, ductal carcinoma tissue, invasive ductal tissue grade 1, invasive ductal tissue grade 2, invasive ductal breast cancer grade 3, and metastases thereof.

33. The kit according to claim 24, wherein said oncology microarray comprises tissue samples from at least five different tumor types.

34. The kit according to claim 33, where said tumor types are selected from the group consisting of colorectal tumors, prostate tumor, lung tumors, breast tumors, kidney tumors, urinary bladder tumors, ovarian tumors, brain tumors, malignant melanoma, and head and neck tumors.

35. A method for detecting the expression of a cancer-specific marker in a test sample, comprising:
(a) providing a test sample comprising cells or tissue;
(b) providing a microarray according to any of claims 1, 3, or 4, wherein said at least one sample comprising abnormally proliferating cells express said cancer-specific marker;
(c) reacting the test sample and the microarray with a molecular probe which specifically reacts with said cancer-specific marker;
(d) detecting the presence, absence or amount of said reactivity in said test sample and comparing said reactivity to the reactivity of said at least one sample.

36. The method according to claim 35, wherein said comparing in step (d) provides a diagnosis or prognosis of cancer in cells or tissue in said test sample.

37. The method according to claim 36, wherein information relating to the reactivity of said molecular probe with said test sample and/or said microarray is entered into a specimen-linked database.

38. The method according to claim 35, wherein said test sample is from a patient treated with a drug.
